(19) United States
(12) Patent Application Publication
(10) Pub. No.: US 2025/0019418 A1
(43) Pub. Date: Jan. 16, 2025

(54) SYSTEMS AND METHODS FOR THE PRODUCTION OF METHEMOGLOBIN AND ITS DERIVATIVES

(71) Applicants: Ohio State Innovation Foundation, Columbus, OH (US); The Regents of the University of California, Oakland, CA (US)

(72) Inventors: Andre PALMER, Columbus, OH (US); Pedro J. CABRALES AREVALO, La Mesa, CA (US); Xiangming GU, Columbus, OH (US)

(21) Appl. No.: 18/708,744

(22) PCT Filed: Sep. 9, 2022
(86) PCT No.: PCT/US22/43124

§ 371 Date: May 9, 2024

Related U.S. Application Data
(60) Provisional application No. 63/277,433, filed on Nov. 9, 2021.

(51) Int. Cl. C07K 14/805; A61K 38/00; C07K 1/113; C07K 1/36

(52) U.S. Cl. CPC C07K 14/805; C07K 1/1136; C07K 1/36; A61K 38/00

(57) ABSTRACT

Disclosed are methods for producing methemoglobin or polymerized methemoglobin. Also provided are pharmaceutical compositions comprising methemoglobin complexed with haptoglobin or polymerized methemoglobin, as well as methods of using thereof to treat cyanide and hydrogen sulfide poisoning.

### CROSS-REFERENCE TO RELATED APPLICATIONS

This application claims the benefit of U.S. Provisional Application No. 63/277,433, filed Nov. 9, 2021, which is hereby incorporated by reference in its entirety.

### STATEMENT REGARDING FEDERALLY SPONSORED RESEARCH OR DEVELOPMENT

This invention was made with government support under grant/contract numbers R01 HL156526, R01 HL138116, R01 HL 126945, R01 EB021926, and R01 HL159862 awarded by the National Institutes of Health. The government has certain rights in the invention.

### BACKGROUND

I vivo exposure to cell-free hemoglobin (Hb) results in adverse side-effects (Buehler, Humar, & Schaer, 2020; Gladwin, Kanias, & Kim-Shapiro, 2012). For experimental studies investigating the toxicity of cell-free Hb, crystalloid solutions such as lactated Ringer's (LR) have been used as the standard control to discern the effects of Hb on physiological responses associated with vasoactivity and oxidative stress (King et al., 2005; Martini, Cortez, & Dubick, 2013; Stern et al., 2009). Unfortunately, LR is not an ideal control material when studying the toxicity of Hb, since it does not possess the same chemical structure or equivalent half-life of cell-free Hb (Hahn & Lyons, 2016; Taguchi, Yamasaki, Maruyama, & Otagiri, 2017). Fortunately, Hb can be converted to methemoglobin (metHb), in which the iron atom is oxidized to the non-functional ferric state (Fe³⁺) from the functional ferrous state (Fe²⁺). Since metHb (HbFe³⁺) is an oxidized form of native Hb (HbFe²⁺), it cannot bind and release gaseous ligands such as oxygen (O₂), nitric oxide (NO) and carbon monoxide (CO) (Mansouri & Lurie, 1993; Van Slyke & Hiller, 1946). Additionally, metHb can be removed from the systemic circulation via haptoglobin (Hp) binding and subsequent CD163 receptor mediated endocytosis as well as via elimination through the kidneys and thus most likely possesses a similar half-life to Hb (Lim, Ferraro, Moore, & Halliwell, 2001). Previously, it was observed in a canine model that the infusion of metHb did not increase mean arterial pressure (MAP) in comparison to cell-free Hb, demonstrating the inability of it to participate in NO signaling pathways (Wang et al., 2013). Therefore, unlike cell-free Hb which can scavenge NO and subsequently elicit vasoconstriction and systemic hypertension, metHb does not elicit those side-effects. Hence, metHb can function as a suitable non-oxygen carrying control material when studying the vasoactive toxicity of cell-free Hb.

MetHb is typically prepared by reacting excess K₃Fe(CN)₆ (on a per heme basis) with Hb (Ford & Ainsworth, 1968; Tomoda & Yoneyama, 1979). The reaction mixture is then separated on a desalting column to remove Fe(CN)₆³⁻ and Fe(CN)₆⁴⁻ from metHb. However, the chromatographic desalting process is not the best approach for bench-top or pilot scale purification of metHb due to sample dilution during the purification process.

Accordingly, improved systems and methods for the preparation of methemoglobin are needed.

### SUMMARY

As described herein, small scale (i.e., 1 mL reaction volume) experiments were initially conducted to determine molar ratios of Hb to the oxidization agents hydrogen peroxide (H₂O₂) and sodium nitrite (NaNO₂) which produce the highest conversion of Hb into metHb. A spectral deconvolution program was then utilized to determine the molar fraction of various species (hemichrome, metHb, oxyHb, metHb-NO₂, and NaNO₂) in solution during the oxidation reaction. From this type of analysis, either a 1:1 or 1:5 molar ratio was identified as a suitable molar ratio of Hb:NaNO₂ (heme basis) that yielded the highest conversion of Hb into metHb with negligible amounts of side products.

A 1:5 molar ratio Hb:NaNO₂ was then used for large scale metHb synthesis, which achieved yields >75% with purities >85% (e.g., a yield of >90% with a purity of >95%, a yield of >90% with a purity of >99%). This reaction was performed using a batch reactor followed by tangential flow filtration to yield purified metHb. The biophysical properties of metHb were then characterized to elucidate the potential of using the synthesized metHb as a non-oxygen carrying control material. The haptoglobin binding kinetics of metHb were found to be similar to Hb. Additionally, the synthesized metHb was stable in phosphate buffered saline (50 mM, pH 7.4) at 4° C. for approximately one week.

MetHb and polymerized metHb can scavenge cyanide ions (CN⁻), and can therefore be used to treat cyanide poisoning. However, metHb-CN is cytotoxic, since it can extravasate from the circulation into the tissue space, where it can deposit cytotoxic iron and cyanide. In contrast polymerized metHb-CN is not as cytotoxic, since it cannot extravasate into the tissue space because of its larger molecular diameter and remains in the circulation to be cleared by tissue resident macrophages and monocytes. It is also possible to stabilize metHb with the plasma protein haptoglobin (Hp) to form the metHb-Hp complex, which can then bind cyanide and facilitate the clearance of the metHb-CN-Hp complex via CD163+ macrophages and monocytes. Therefore, polymerized metHb and the metHb-Hp complex can be administered as cyanide scavengers to treat cyanide poisoning. Similarly, polymerized metHb and the metHb-Hp complex can be administered as hydrogen sulfide (HS⁻) scavengers to treat hydrogen sulfide poisoning.

Accordingly, described herein are methods and systems for the production of methemoglobin, polymerized methemoglobin and methemoglobin-haptoglobin complexes. These methods are readily scalable can efficiently produce methemoglobin, polymerized methemoglobin, and the methemoglobin-haptoglobin complex at yields of greater than 90% and purities of greater than 95% (e.g., greater than 99%). These methods can comprise (i) contacting a solution comprising ferrous hemoglobin or polymerized ferrous hemoglobin with an oxidizing agent under conditions effective to produce a solution comprising methemoglobin or polymerized methemoglobin; and (ii) filtering the solution comprising methemoglobin or polymerized methemoglobin by ultrafiltration against a filtration membrane having a pore size that separates the protein from the oxidizing agent and reaction byproducts, thereby forming a retentate fraction comprising the methemoglobin or polymerized methemoglobin and a permeate fraction comprising impurities. The methemoglobin can be further reacted with haptoglobin to form the methemoglobin-haptoglobin complex, followed by filtering the solution to remove unbound reactants.

In some embodiments, the ferrous hemoglobin or polymerized ferrous hemoglobin is from a mammalian, invertebrate, or recombinant source. In certain embodiments, the ferrous hemoglobin or polymerized ferrous hemoglobin is from a mammalian source. For example, the ferrous hemoglobin or polymerized ferrous hemoglobin can comprise bovine hemoglobin, procine hemoglobin, or human hemoglobin. In certain embodiments, the ferrous hemoglobin or polymerized ferrous hemoglobin can comprise recombinantly produced hemoglobin. In other embodiments, the ferrous hemoglobin or polymerized ferrous hemoglobin can comprise chemically or genetically modified hemoglobin that, for example, prevent dissociation of the hemoglobin molecule, or modify the oxygen-binding affinity, or modify hemoglobin binding with haptoglobin. The polymerized ferrous hemoglobin can comprise any suitable polymerized hemoglobin. The polymerized ferrous hemoglobin can be in the tense or relaxed quaternary state, or in between these two quaternary states.

In some embodiments, the solution comprising ferrous hemoglobin or polymerized ferrous hemoglobin comprises an aqueous solution buffered at a pH of from 6 to 8.

In some embodiments, step (i) comprises combining the ferrous hemoglobin or polymerized ferrous hemoglobin with the oxidizing agent in a reactor. The oxidizing agent can comprise any suitable oxidizing agent, such as sodium nitrite, potassium nitrite, an inorganic peroxide, an organic peroxide, or any combination thereof. In certain embodiments, the oxidizing agent can comprise sodium nitrite, potassium nitrite, or any combination thereof.

In some embodiments, step (i) can comprise combining the ferrous hemoglobin or polymerized ferrous hemoglobin with the oxidizing agent (e.g., sodium nitrite, potassium nitrite, or any combination thereof) at a molar ratio (heme basis) of from 2:1 to 1:10, such as from 1:1 to 1:5.

In some embodiments, step (i) can comprise reacting the ferrous hemoglobin or polymerized ferrous hemoglobin with the oxidizing agent for from 1 minute to 24 hours, such as from 5 minutes to 8 hours or from 5 minutes to 4 hours.

In some embodiments, step (i) can comprise reacting the ferrous hemoglobin or polymerized ferrous hemoglobin with the oxidizing agent at a temperature of from 1° C. to 40° C. (e.g., 18° C. to 40° C.).

The ultrafiltration process in step (ii) can comprise, for example, tangential flow filtration or cross-flow filtration.

The filtration membrane can be rated for removing solutes having a molecular weight less than the molecular weight of the methemoglobin or polymerized methemoglobin. For example, in some embodiments, the filtration membrane is rated for removing solutes having a molecular weight of from 1 to 750 kDa, such as from 1 to 500 kDa, from 1 to 250 kDa, from 1 to 100 kDa, from 1 to 50 kDa, or from 1 to 10 kDa.

In some embodiments, filtering step (ii) can comprise buffer exchange.

In some embodiments, filtering step (ii) can comprise continuous diafiltration or dialysis. If desired, the retentate fraction can be spectroscopically monitored during the continuous diafiltration to monitor the formation of methemoglobin or polymerized methemoglobin, the concentration of methemoglobin or polymerized methemoglobin, the concentration of an impurity, or any combination thereof.

In some embodiments, the methemoglobin or polymerized methemoglobin produced using the methods described herein (e.g., present in the retentate fraction) is stable at 4° C. in PBS (0.1 M, pH 7.4) for a period of at least 7 days.

Methemoglobin and polymerized methemoglobin produced using the methods described herein can be incorporated in pharmaceutical compositions. Accordingly, also provided are pharmaceutical compositions comprising methemoglobin or polymerized methemoglobin prepared using the methods described herein. In some embodiments, the pharmaceutical composition can comprise methemoglobin, and the methemoglobin is complexed with haptoglobin. In some embodiments, the methemoglobin is complexed with haptoglobin at a weight ratio of from 1:1 to 1:3 (methemoglobin:haptoglobin). In some embodiments, the haptoglobin has an average molecular weight of from 80 kDa to 1,000 kDa, such as from 100 kDa to 1,000 kDa.

In some embodiments, the methemoglobin, the polymerized methemoglobin, or the methemoglobin can be complexed with haptoglobin is conjugated to a therapeutic or diagnostic agent. In some embodiments, the therapeutic or diagnostic agent comprises an imaging agent, such as an MRI contrast agent, a porphyrin-based imaging agent, and/or a phthalocyanine-based imaging agent, a porphyrin-based photodynamic therapy agent, a phthalocyanine-based photodynamic therapy agent, an agent to treat or prevent a disease or disorder associated with the overexpression of CD163, an agent to treat or prevent a disease which involves macrophages or monocytes, an anti-cancer agent, an anti-inflammatory agent, an agent that treats or prevents an infection, hydrogen sulfide, or a combination thereof.

These pharmaceutical compositions can be administered to a subject in need thereof to treat cyanide or hydrogen sulfide poisoning. The compositions can also be used to deliver active agents to CD163+ macrophages and monocytes (e.g., as an immunotherapy targeting CD163+ macrophages and monocytes). Compositions comprising hydrogen sulfide bound to the methemoglobin, the polymerized methemoglobin, or the methemoglobin complexed with haptoglobin can also be used to deliver a subtoxic dose of hydrogen sulfide to lower the metabolism of a cell, tissue, organ, or subject.

### DESCRIPTION OF DRAWINGS

FIG. 1A schematically illustrates a reactor and fluid recirculation system used to synthesize metHb.

FIG. 1B schematically illustrates a tangential flow filtration system used to purify metHb.

FIG. 2 illustrates the oxidization of Hb at varying Hb:oxidization agent molar ratios (heme basis, 1:1, 1:5, and 1:10). The oxidation of 60 μM Hb (heme basis) with either NaNO₂ or H₂O₂ was monitored via UV-visible spectrometry in 0.1 M PBS pH 7.4 at room temperature for 21 h. Since multiple Hb species were observed, a deconvolution program was used to determine the composition of each Hb species within the reaction mixture at each time point.

FIG. 3A shows SEC-HPLC chromatograms of native Hb and metHb after TFF processing monitored at λ=420 nm prepared using a sample of bovine Hb (bHb). SEC-HPLC was performed using a Thermo Scientific Dionex UltiMate 3000 UHPLC/HPLC system coupled with an Acclaim SEC-1000 column.

FIG. 3B shows SEC-HPLC chromatograms of native Hb and metHb after TFF processing monitored at λ=420 nm prepared using a sample of human Hb (hHb). SEC-HPLC was performed using a Thermo Scientific Dionex UltiMate 3000 UHPLC/HPLC system coupled with an Acclaim SEC-1000 column.

FIG. 3C shows a comparison of the absorption spectra of oxygenated bHb (oxybHb) before synthesis and bovine metHb (metbHb) after synthesis and TFF processing.

FIG. 3D shows a comparison of the absorption spectra of oxygenated hHb (oxyhHb) before synthesis and human metHb (methHb) after synthesis and TFF processing.

FIGS. 4A-4B illustrate the composition of bovine metHb (FIG. 4A, metbHb, 1.5 L reactor system) and human metHb (FIG. 4B, methHb, 1.5 L reactor system) during TFF processing. Stage 0: Both metbHb and methHb were initially transferred into a 2 L Nalgene bottle and concentrated to 500 mL (total volume). Stage 1: The solution was then subject to constant volume diafiltration on a 50 kDa hollow fiber (HF) module within a 500 mL bottle. Stage 3: The solution at the end of Stage 1 was further concentrated to ˜100 mg/mL and stored at −80° C.

FIG. 5A is a plot showing the pseudo first order Hp binding kinetics of Hb/metHb. Excess Hb/metHb was rapidly mixed with Hp (0.25 μM on a Hb tetramer binding basis). The normalized fluorescence changes were fit to a monoexponential equation to regress the pseudo first order Hp binding rate constant.

FIG. 5B is a plot showing the dependence of the pseudo first order Hp binding rate constant as a function of Hb/metHb concentration. The second order Hp binding rate constant was obtained by performing a linear fit of the pseudo first order Hp binding rate constant to the Hb/metHb concentration.

FIG. 6 is a plot showing the SEC-HPLC of Hp-metHb/Hb complexes as a function of metHb/Hb concentration. Panels A-D show the normalized SEC-HPLC chromatograms of Hp-metbHb (panel A), Hp-bHb (panel B), Hp-methHb (panel C), and (D) Hp-hHb mixtures (panel D). Chromatograms were monitored at an absorbance of λ=413 nm normalized against the total area under the curve. Hp (0.25 μM on a Hb tetramer binding basis) was rapidly mixed with excess metHb/Hb. Panels E-H show the composition of Hp-metbHb (panel E), Hp-bHb (panel F), Hp-methHb (panel G), and Hp-hHb (panel H) mixtures which include the Hp-metHb/Hb complex, metHb/Hb tetramer (α₂β₂), metHb/Hb dimer (αβ) and monomer (α/β) as a function of metHb/Hb concentration.

FIG. 7 is a plot showing the SEC-HPLC of metHb/Hb as a function of metHb/Hb concentration to monitor metHb/Hb dissociation after 1 week of storage at 4° C. Panels A-D show normalized intensity SEC-HPLC chromatograms of metbHb (panel A), bHb (panel B), methHb (panel C), and hHb (panel D). Chromatograms were monitored at an absorbance of λ=413 nm normalized against the total area under the curve. Panels E-H show the composition of metbHb (panel E), bHb (panel F), methHb (panel G), and hHb (panel H) species including the metHb/Hb tetramer (α₂β₂), metHb/Hb dimer (αβ) and monomer (α/β) as a function of metHb/Hb concentration.

FIGS. 8A-8D are plots showing the equilibria between dimers and tetramers of metbHb (FIG. 8A), bHb (FIG. 8B), methHb (FIG. 8C), and hHb (FIG. 8D). The tetramer-dimer dissociation constant (K_{d}) shown in each figure inset was calculated as described in the text. All samples were incubated in PBS (0.1 M, pH 7.4) and analyzed on a Thermo Scientific Dionex UltiMate 3000 UHPLC/HPLC system coupled with an Acclaim SEC-1000 column. Deconvolution analysis was then performed on the chromatogram of all samples to yield the fraction of Hb tetramers.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (i.e. filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is processed (e.g., continually processed) downstream.

As used herein, the term “ultrafiltration” is used for processes employing membranes rated for retaining solutes having a molecular weight between about 1 kDa and 1000 kDa.

As used herein, the term “reverse osmosis” refers to processes employing membranes capable of retaining solutes of a molecular weight less than 1 kDa such as salts and other low molecular weight solutes.

As used herein, the term “microfiltration” refers to processes employing membranes in the 0.1 to 10 micron pore size range.

As used herein, the expression “transmembrane pressure” or “TMP” refers to the pressure differential gradient that is applied along the length of a filtration membrane to cause fluid and filterable solutes to flow through the filter.

The term “hydrophobic,” as used herein, refers to a ligand which, as a separate entity, exhibits a higher solubility in a non-aqueous solution (e.g., octanol) than in water.

The term “conjugated protein,” as used herein, refers to a protein complex that includes an apoprotein and one or more associated hydrophobic ligands. The one or more hydrophobic ligands may by covalently or non-covalently associated with the apoprotein. Examples of conjugated proteins include, for example, lipoproteins, glycoproteins, phosphoproteins, hemoproteins, flavoproteins, metalloproteins, phytochromes, cytochromes, opsins, and chromoproteins.

The phrase “mild denaturing,” as used herein refers to a process which reversibly disrupts the secondary, tertiary, and/or quaternary structure of the conjugated protein, thereby facilitating separation of the hydrophobic ligand from the apoprotein. Mild denaturing can be distinguished from harsher conditions, which cleave the peptide backbone, primarily produce insoluble protein upon denaturation/renaturation, and/or disrupt protein structure to a degree such that the protein loses its biological function upon refolding.

The terms “isolating,” “purifying,” and “separating,” as used interchangeably herein, refer to increasing the degree of purity of a polypeptide or protein of interest or a target protein from a composition or sample comprising the polypeptide and one or more impurities (e.g., additional proteins or polypeptides).

The term “haptoglobin” as used herein refers to a protein that is synthesized and secreted mainly in the liver. In blood plasma, haptoglobin (Hp) binds to cell-free hemoglobin (Hb) released from erythrocytes with high affinity and thereby inhibits Hb oxidative activity. The Hp-Hb complex is then removed by the reticuloendothelial system (mostly in the spleen and liver). Hp, in its simplest form, consists of two alpha-beta dimer chains, connected by disulfide bridges, but can exist as polymeric alpha-beta dimer species. The chains originate from a common precursor protein, which is proteolytically cleaved during protein synthesis. Hp exists in two allelic forms in the human population, so-called Hp1 and Hp2, the latter one having arisen due to partial duplication of the Hp1 gene. Three genotypes of Hp, therefore, are found in humans: Hp1-1, Hp2-1, and Hp2-2. Hp of different genotypes have been shown to have similar effects in vivo in attenuating Hb-mediated toxicity. Furthermore, a protein with >90% sequence identity to the Hp1 gene, called haptoglobin related protein (Hpr) also has high affinity for Hb. The term “haptoglobin” thus encompasses all Hp phenotypes (Hp1-1,Hp2-2 and Hp2-1).

### Methods, Compositions, and Methods of Use

Described herein are methods and systems for the production of methemoglobin and polymerized methemoglobin. These methods are readily scalable can efficiently produce methemoglobin and polymerized methemoglobin at yields of greater than 75% (e.g., greater that 85%, or greater than 90%) and purities of greater than 85% (e.g., greater than 90%, greater than 95%, or greater than 99%). These methods can comprise (i) contacting a solution comprising ferrous hemoglobin or polymerized ferrous hemoglobin with an oxidizing agent under conditions effective to produce a solution comprising methemoglobin or polymerized methemoglobin; and (ii) filtering the solution comprising methemoglobin or polymerized methemoglobin by ultrafiltration against a filtration membrane having a pore size that separates the protein from the oxidization agent and reaction byproducts, thereby forming a retentate fraction comprising the methemoglobin or polymerized methemoglobin and a permeate fraction comprising impurities.

The ferrous hemoglobin or polymerized ferrous hemoglobin can be any suitable ferrous heme-based oxygen carrier.

Hemoglobin (Hb) is the oxygen-carrying component of blood that circulates through the bloodstream inside small enucleate cells known as erythrocytes or red blood cells. It is a protein comprised of four associated polypeptide chains that bear prosthetic groups known as hemes. The structure of hemoglobin is well known and described in Bunn & Forget, eds., Hemoglobin: Molecular, Genetic and Clinical Aspects (W. B. Saunders Co., Philadelphia, Pa.: 1986) and Fermi & Perutz “Hemoglobin and Myoglobin,” in Phillips and Richards, Atlas of Molecular Structures in Biology (Clarendon Press: 1981). As blood circulates through the lungs, the oxygen present in the alveolar capillaries diffuses through the alveolar membrane and acts to convert virtually all of the hemoglobin within the red cells to a reversible molecular complex known as oxyhemoglobin. During this oxygenation process, the red blood cells become cherry red in color. Because the association of the oxygen and hemoglobin molecules within the red cells is reversible, the oxygen molecules are gradually released from the hemoglobin molecules (or from the red blood cells) when blood reaches the tissue capillaries. Eventually, the oxygen molecules diffuse into the tissues and is consumed by metabolism. As the oxyhemoglobin releases its bound oxygen, the red cells become purple in color.

As used herein, the term “hemoglobin” refers to the iron-containing oxygen-transport metalloprotein in the red blood cells of all vertebrates. Hemoglobin used in the present invention can be obtained from a variety of mammalian sources, such as, for example, human, or bovine (genus bos), or bison (genus bison), or ovine (genus ovis), or porcine (genus sus) sources, or other vertebrates or as transgenically-produced hemoglobin. Alternatively, the hemoglobin for use in the methods and compositions described herein can be synthetically produced by a bacterial cell, or more preferably, by a yeast cell, mammalian cell, or insect cell expression system (Hoffman, S. J. et al., U.S. Pat. No. 5,028,588 and Hoffman, et al., WO 90/13645, both herein incorporated by reference). Alternatively, hemoglobin can be obtained from transgenic animals; such animals can be engineered to express non-endogenous hemoglobin (Logan, J. S. et al. PCT Application No. PCT/US92/05000; Townes, T. M. et al., PCT Application No. PCT/US/09624, both herein incorporated by reference in their entirety).

Hemoglobin can also encompass genetically modified and/or recombinantly produced hemoglobin as well as chemically treated or surface decorated hemoglobins either in their dimeric, or tetrameric or variously polymerized forms. Expression of various recombinant hemoglobins has been achieved. Such expression methods include individual globin expression as described, for example, in U.S. Pat. No. 5,028,588, and di-alpha globin expression created by joining two alpha globins with a glycine linker through genetic fusion coupled with expression of a single beta globin gene to produce a pseudotetrameric hemoglobin molecule as described in WO 90/13645 and Looker et al., Nature 356:258 260 (1992). Other modified recombinant hemoglobins are disclosed in PCT Publication WO 96/40920. Similar to other heterologous proteins expressed in E. coli, recombinant hemoglobins have N-terminal methionines, which in some recombinant hemoglobins replace the native N-terminal valines.

In some embodiments, the ferrous hemoglobin or polymerized ferrous hemoglobin is from a mammalian, invertebrate, or recombinant source. In certain embodiments, the ferrous hemoglobin or polymerized ferrous hemoglobin is from a mammalian source. For example, the ferrous hemoglobin or polymerized ferrous hemoglobin can comprise bovine hemoglobin, procine hemoglobin, or human hemoglobin. In certain embodiments, the ferrous hemoglobin or polymerized ferrous hemoglobin can comprise recombinantly produced hemoglobin. In other embodiments, the ferrous hemoglobin or polymerized ferrous hemoglobin can comprise chemically or genetically modified hemoglobin that, for example, prevent dissociation of the hemoglobin molecule or modify the oxygen-binding affinity.

In some embodiments the solution can comprises polymerized ferrous hemoglobin. The term “polymerized,” as used herein, encompasses both inter-molecular and intramolecular polyhemoglobin, with at least 50%, preferably greater than about 95%, of the polymerized hemoglobin of greater than the tetrameric form. The polymerized hemoglobin can be prepared by polymerizing or cross-linking hemoglobin with a multifunctional cross-linking agent. Preferably, the polymerized hemoglobin is substantially soluble in aqueous fluids having a pH of 6 to 9 and in physiological fluids. Suitable examples of cross-linking agents are disclosed in U.S. Pat. No. 4,001,200, the entire teachings of which are incorporated herein by reference.

Suitable specific examples of the cross-linking agents include compounds having an aldehyde or dialdehyde functionality, such as formaldehyde, paraformaldehyde, glutaraldehyde, formaldehyde activated ureas such as 1,3-bis(hydroxymethyl)urea, N,N′-di(hydroxymethyl) imidazolidinone prepared from formaldehyde condensation with a urea; compounds bearing a functional isocyanate or isothiocyanate group, such as diphenyl-4,4′-diisothiocyanate-2,2′-disulfonic acid, toluene diisocyanate, toluene-2-isocyanate-4-isothiocyanate, 3-methoxydiphenylmethane-4,4′-diisocyanate, propylene diisocyanate, butylene diisocyanate, and hexamethylene diisocyanate; esters and thioesters activated by strained thiolactones; hydroxysuccinimide esters; halogenated carboxylic acid esters; and imidates. Other examples of the cross-linking agents include derivatives of carboxylic acids and carboxylic acid residues of hemoglobin activated in situ to give a reactive derivative of hemoglobin that will cross-link with the amines of another hemoglobin. Examples of the carboxylic acids include citric, malonic, adipic and succinic acids. Carboxylic acid activators include thionyl chloride, carbodiimides, N-ethyl-5-phenyl-isoxazolium-3′-sulphonate (Woodward's reagent K), N,N′-carbonyldiimidazole, N-t-butyl-5-methylisoxazolium perchlorate (Woodward's reagent L), 1-ethyl-3-dimethyl aminopropylcarbodiimde, and 1-cyclohexyl-3-(2-moφholinoethyl) carbodiimide metho-p-toluene sulfonate. The cross-linking reagent can be a dialdehyde precursor that readily forms a bifunctional dialdehyde in the reaction medium. Suitable dialdehyde precursors include acrolein dimer or 3,4-dihydro-1,2-pyran-2-carboxaldehyde which undergoes ring cleavage in an aqueous environment to give alpha-hydroxy-adipaldehyde. Other precursors, which on hydrolysis yield a cross-linking reagent, include 2-ethoxy-3,4-dihydro-1,2-pyran which gives glutaraldehyde, 2-ethoxy-4-methyl-3,4-dihydro-1,2-pyran which yields 3-methyl glutaraldehyde, 2,5-diethoxy tetrahydrofuran which yields succinic dialdehyde and 1,1,3,3-tetraethoxypropane which yields malonic dialdehyde and formaldehyde from trioxane. Exemplary commercially-available cross-linking reagents include divinyl sulfone, epichlorohydrin, butadiene diepoxide, ethylene glycol diglycidyl ether, glycerol diglycidyl ether, dimethyl suberimidate dihydrochloride, dimethyl malonimidate dihydrochloride, and dimethyl adipimidate dihydrochloride.

Specific examples of the cross-linking agents include glutaraldehyde, succindialdehyde, activated forms of polyoxyethylene and dextran, cv-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class or the aryl dihalide class.

In some embodiments, the polymerized hemoglobin can comprise hemoglobin polymerized by a dialdehyde. As used herein, the “hemoglobin polymerized by a dialdehyde” includes both hemoglobin polymerized by a dialdehyde and hemoglobin polymerized by a dialdehyde precursor that readily forms a bifunctional dialdehyde in the reaction medium. Suitable dialdehyde and dialdehyde precursors are as described above. In some embodiments, the polymerized hemoglobin can comprise hemoglobin polymerized by glutaraldehyde.

The polymerized ferrous hemoglobin can be in the tense or relaxed quaternary state, or in between these two quaternary states.

In some embodiments, the ferrous hemoglobin or polymerized ferrous hemoglobin can be encapsulated and/or in the form of nanoparticles.

In some embodiments, the ferrous hemoglobin or polymerized ferrous hemoglobin can be surface-conjugated (e.g., PEGylated hemoglobin).

In some embodiments, the solution comprising ferrous hemoglobin or polymerized ferrous hemoglobin comprises an aqueous solution buffered at a pH of from 6 to 8 (e.g., from 6.5 to 7.5, or from 6.8 to 7.2).

In some embodiments, step (i) comprises combining the ferrous hemoglobin or polymerized ferrous hemoglobin with the oxidizing agent in a reactor. The oxidizing agent can comprise any suitable oxidizing agent, such as sodium nitrite, potassium nitrite, an inorganic peroxide, an organic peroxide, or any combination thereof. In certain embodiments, the oxidizing agent can comprise sodium nitrite, potassium nitrite, or any combination thereof.

In some embodiments, step (i) can comprise combining the ferrous hemoglobin or polymerized ferrous hemoglobin with the oxidizing agent (e.g., sodium nitrite, potassium nitrite, or any combination thereof) at a molar ratio (heme basis) of from 2:1 to 1:10, such as from 1:1 to 1:5.

In some embodiments, step (i) can comprise reacting the ferrous hemoglobin or polymerized ferrous hemoglobin with the oxidizing agent for from 1 minute to 24 hours, such as from 5 minutes to 8 hours or from 5 minutes to 4 hours.

In some embodiments, step (i) can comprise reacting the ferrous hemoglobin or polymerized ferrous hemoglobin with the oxidizing agent at a temperature of from 1° C. to 40° C. (e.g., 18° C. to 40° C.).

Generally, the filtration membrane can have a range of pore sizes effective to separate the methemoglobin or polymerized methemoglobin from impurities (e.g., a pore size which allows the impurities to pass through the filtration membrane but retains the methemoglobin or polymerized methemoglobin). For example, the filtration membrane can be rated for removing solutes having a molecular weight less than the molecular weight of the methemoglobin or polymerized methemoglobin. For example, in some embodiments, the filtration membrane is rated for removing solutes having a molecular weight of from 1 to 750 kDa, such as from 1 to 500 kDa, from 1 to 250 kDa, from 1 to 100 kDa, from 1 to 50 kDa, or from 1 to 10 kDa.

In connection with the methods described herein, ultrafiltration can comprise direct-flow filtration (DFF), cross-flow or tangential-flow filtration (TFF), or a combination thereof. In certain embodiments, the ultrafiltration can comprise tangential-flow filtration (TFF).

The membranes useful in the filtration steps described herein can be in the form of flat sheets, rolled-up sheets, cylinders, concentric cylinders, ducts of various cross-section and other configurations, assembled singly or in groups, and connected in series or in parallel within the filtration unit. The apparatus can be constructed so that the filtering and filtrate chambers run the length of the membrane.

Suitable membranes include those that separate the desired species from undesirable species in the mixture without substantial clogging problems and at a rate sufficient for continuous operation of the system. Examples are described, for example, in Gabler FR. Tangential flow filtration for processing cells, proteins, and other biological components.ASM News 1984; 50:299-304. They can be synthetic membranes of either the microporous type or the ultrafiltration type. A microporous membrane has pore sizes typically from 0.1 to 10 micrometers, and can be made so that it retains all particles larger than the rated size. Ultrafiltration membranes have smaller pores and are characterized by the size of the protein that will be retained. They are available in increments from 1000 to 1,000,000 Dalton nominal molecular weight limits.

Generally, the filtration membrane can comprise an ultrafiltration membrane. Ultrafiltration membranes are normally asymmetrical with a thin film or skin on the upstream surface that is responsible for their separating power. They are commonly made of regenerated cellulose, polysulfone or polyethersulfone. In some cases, the filtration membrane can be rated for retaining solutes having a molecular weight of from 1 to 750 kDa, such as from 1 to 500 kDa, from 1 to 250 kDa, from 1 to 100 kDa, or from 1 to 50 kDa.

In some cases, each filtration step can involve filtration through a single filtration membrane. In other cases, because membrane filters are not perfect and may have holes that allow some intended retentate molecules to slip through, more than one membrane (e.g., two membranes, three membranes, four membranes, or more) having the same pore size can be utilized for a given filtration step. In these embodiments, the membranes can be placed so as to be layered parallel to each other (e.g., one on top of the other) such that filtered fluid sequentially flows through each of the more than one membrane.

Membrane filters for tangential-flow filtration are available as units of different configurations depending on the volumes of liquid to be handled, and in a variety of pore sizes. Particularly suitable for use in the methods described herein, on a relatively large scale, are those known, commercially available tangential-flow filtration units.

The filtration unit useful herein is suitably any unit now known or discovered in the future that serves as an appropriate filtration module, particularly for microfiltration and ultrafiltration. The preferred filtration unit is hollow fibers or a flat sheet device. These sandwiched filtration units can be stacked to form a composite cell. One example type of rectangular filtration plate type cell is available from Filtron Technology Corporation, Northborough, Mass., under the trade name Centrasette. Another example filtration unit is the Millipore Pellicon ultrafiltration system available from Millipore, Bedford, Mass.

In some embodiments, filtering step (ii) can comprise buffer exchange.

In some embodiments, filtering step (ii) can comprise continuous diafiltration or dialysis. If desired, the retentate fraction can be spectroscopically monitored during the continuous diafiltration to monitor the formation of methemoglobin or polymerized methemoglobin, the concentration of methemoglobin or polymerized methemoglobin, the concentration of an impurity, or any combination thereof.

The purity of isolated methemoglobin or polymerized methemoglobin can be assessed using a variety of methods known in the art, including for example, liquid chromatography and/or spectroscopic methods (UV-visible spectroscopy, fluorescence spectroscopy, etc.). In certain embodiments, the methemoglobin or polymerized methemoglobin isolated in step (ii) can comprise less than 2% (e.g., less than 1.5%, less than 1%, less than 0.75%, less than 0.5%, less than 0.25%, or less than 0.1%) residual impurities relative to the concentration of methemoglobin or polymerized methemoglobin isolated in step (ii), as measured by a suitable spectroscopic method (e.g., SEC-HPLC coupled with UV-visible spectroscopy).

In some embodiments, the methemoglobin or polymerized methemoglobin prepared using the methods described herein (e.g., present in the retentate fraction) is stable at 4° C. in PBS (0.1 M, pH 7.4) for a period of at least 7 days.

Methemoglobin and polymerized methemoglobin produced using the methods described herein can be incorporated in pharmaceutical compositions. Accordingly, also provided are pharmaceutical compositions comprising methemoglobin or polymerized methemoglobin prepared using the methods described herein.

In some embodiments, the pharmaceutical composition can comprise methemoglobin, and the methemoglobin is complexed with haptoglobin.

In some embodiments, the methemoglobin-haptaglobin complex can comprise methhemoglobin and haptoglobin (Hp) at a weight ratio of at least 1:1 (e.g., at least 1:1.1, at least 1:1.2, at least 1:1.3, at least 1:1.4, at least 1:1.5, at least 1:1.6, at least 1:1.7, at least 1:1.8, at least 1:1.9, at least 1:2, at least 1:2.1, at least 1:2.2, at least 1:2.3, at least 1:2.4, at least 1:2.5, at least 1:2.6, at least 1:2.7, at least 1:2.8, at least 1:2.9 or at least 1:3). In some embodiments, the methemoglobin-haptaglobin complex can comprise methemoglobin and Hp at a weight ratio of 1:3 or less (e.g., 1:2.9 or less, 1:2.8 or less, 1:2.7 or less, 1:2.6 or less, 1:2.5 or less, 1:2.4 or less, 1:2.3 or less, 1:2.2 or less, 1:2.1 or less, 1:2 or less, 1:1.9 or less, 1:1.8 or less, 1:1.7 or less, 1:1.6 or less, 1:1.5 or less, 1:1.4 or less, 1:1.3 or less, 1:1.2 or less, or 1:1.1 or less).

The methemoglobin-haptaglobin complex comprises methemoglobin and Hp at a weight ratio ranging from any of the minimum values described above to any of the maximum values described above. For example, in some embodiments, the methemoglobin-haptaglobin complex can comprise methemoglobin and Hp at a weight ratio of from 1:1 to 1:3 (e.g., from 1:1.5 to 1:2.5, or from 1:1.7 to 1:2.2, or from 1:2.5 to 1:3).

In some embodiments, the Hp can be prepared using the ultrafiltration methods described below. For example, Hp can be prepared from plasma or fraction thereof (e.g., plasma fraction IV, plasma fraction V, a fraction of precipitated plasma (from salting out, or equivalent) or a combination thereof).

In certain embodiments, the Hp can have an average molecular weight of at least 70 kDa (e.g., at least 80 kDa, at least 90 kDa, at least 100 kDa, at least 150 kDa, at least 200 kDa, at least 250 kDa, at least 300 kDa, at least 350 kDa, at least 400 kDa, at least 450 kDa, at least 500 kDa, at least 550 kDa, at least 600 kDa, at least 650 kDa, at least 700 kDa, at least 750 kDa, at least 800 kDa, at least 850 kDa, at least 900 kDa, or at least 950 kDa). In certain embodiments, the Hp can have an average molecular weight of 1,000 kDa or less (e.g., 950 kDa or less, 900 kDa or less, 850 kDa or less, 800 kDa or less, 750 kDa or less, 700 kDa or less, 650 kDa or less, 600 kDa or less, 550 kDa or less, 500 kDa or less, 450 kDa or less, 400 kDa or less, 350 kDa or less, 300 kDa or less, 250 kDa or less, 200 kDa or less, 150 kDa or less, or 100 kDa or less).

The Hp can have an average molecular weight ranging from any of the minimum values described above to any of the maximum values described above. For example, in some embodiments, the Hp can have an average molecular weight of from 70 kDa to 1,000 kDa (e.g., from 80 kDa to 1,000 kDa, from 90 kDa to 800 kDa, from 80 kDa to 1,000 kDa, or from 80 kDa to 800 kDa).

The methemoglobin-haptaglobin complex can be formed by combining methemoglobin and Hp at an appropriate weight ratio. Following complexation, the methemoglobin-haptaglobin complex can be purified using tangential flow filtration, if desired (e.g., diafiltration using a 70 kDa TFF module to remove excess methemoglobin).

In some embodiments, the methemoglobin, the polymerized methemoglobin, or the methemoglobin can be complexed with haptoglobin is conjugated to an active agent (e.g., a therapeutic or diagnostic agent). In some cases, the active agent can be non-covalently associated with the methemoglobin, the polymerized methemoglobin, or the methemoglobin can be complexed with haptoglobin. For example, in some cases, the active agent can be a hydrophobic active agent that non-covalently associates with the methemoglobin, the polymerized methemoglobin, or the methemoglobin can be complexed with haptoglobin. In other cases, the active agent can be covalently attached the methemoglobin, the polymerized methemoglobin, or the methemoglobin can be complexed with haptoglobin.

In certain embodiments, active agent can be covalently tethered to the the methemoglobin, the polymerized methemoglobin, or the methemoglobin can be complexed with haptoglobin via a linking group. When present, the linking group can be any suitable group or moiety which is at minimum bivalent, and connects the active agent to the protein. The linking group can be composed of any assembly of atoms, including oligomeric and polymeric chains. In some cases, the total number of atoms in the linking group can be from 3 to 200 atoms (e.g., from 3 to 150 atoms, from 3 to 100 atoms, from 3 and 50 atoms, from 3 to 25 atoms, from 3 to 15 atoms, or from 3 to 10 atoms).

In some embodiments, the linking group can be, for example, an alkyl, alkoxy, alkylaryl, alkylheteroaryl, alkylcycloalkyl, alkylheterocycloalkyl, alkylthio, alkylsulfinyl, alkylsulfonyl, alkylamino, dialkylamino, alkylcarbonyl, alkoxycarbonyl, alkylaminocarbonyl, dialkylaminocarbonyl, or polyamino group. In some embodiments, the linking group can comprise one of the groups above joined to one or both of the moieties to which it is attached by a functional group. Examples of suitable functional groups include, for example, secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, or —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. For example, in some embodiments, the linking group can comprise an alkyl group (e.g., a C₁-C₁₂ alkyl group, a C₁-C₈ alkyl group, or a C₁-C₆ alkyl group) bound to one or both of the moieties to which it is attached via an ester (—COO—, —CH₂O₂C—, CHRO₂C—), a secondary amide (—CONH—), or a tertiary amide (—CONR—), wherein R is an alkyl group, an aryl group, or a heterocyclic group. In certain embodiments, the linking group can be chosen from one of the following.

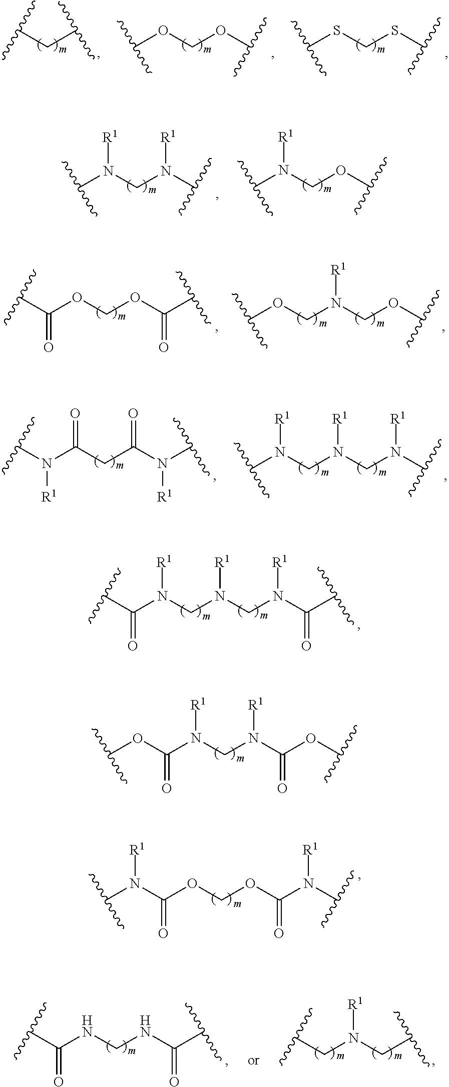

where m is an integer from 1 to 12 and R¹ is, independently for each occurrence, hydrogen, an alkyl group, an aryl group, or a heterocyclic group.

If desired, the linker can serve to modify the solubility of the methemoglobin, the polymerized methemoglobin, or the methemoglobin can be complexed with haptoglobin. In some embodiments, the linker can be hydrophilic. In some embodiments, the linker can be an alkyl group, an alkylaryl group, an oligo- or polyalkylene oxide chain (e.g., an oligo- or polyethylene glycol chain), or an oligo- or poly(amino acid) chain.

In certain embodiments, the linker can be cleavable (e.g., cleavable by hydrolysis under physiological conditions, enzymatically cleavable, or a combination thereof). Examples of cleavable linkers include a hydrolysable linker, a pH cleavage linker, an enzyme cleavable linker, or disulfide bonds that are cleaved through reduction by free thiols and other reducing agents; peptide bonds that are cleaved through the action of proteases and peptidase; nucleic acid bonds cleaved through the action of nucleases; esters that are cleaved through hydrolysis either by enzymes or through the action of water in vivo; hydrazones, acetals, ketals, oximes, imine, aminals and similar groups that are cleaved through hydrolysis in the body; photo-cleavable bonds that are cleaved by the exposure to a specific wavelength of light; mechano-sensitive groups that are cleaved through the application of ultrasound or a mechanical strain (e.g., a mechanical strain created by a magnetic field on a magneto-responsive gel).

The active agent can comprise any suitable therapeutic or diagnostic agent.

In some embodiments, the therapeutic agent can comprise a diagnostic agent (e.g., an imaging agent, such as an MRI contrast agent). Suitable diagnostic agents can include molecules that are detectable in the body of a subject by an imaging technique such as X-ray radiography, ultrasound, computed tomography (CT), single-photon emission computed tomography (SPECT), magnetic resonance imaging (MRI), positron emission tomography (PET), optical fluorescent imaging, optical visible light imaging, and nuclear medicine including Cerenkov light imaging. For example, the diagnostic agent can comprise a radionuclide, paramagnetic metal ion, or a fluorophore.

In some cases, the diagnostic agent can comprise a metal chelator. The terms “metal chelator” and “chelating agent” refer to a polydentate ligand that can form a coordination complex with a metal atom. It is generally preferred that the coordination complex is stable under physiological conditions. That is, the metal will remain complexed to the chelator in vivo.

In some cases, the metal chelator is a molecule that complexes to a radionuclide metal or paramagnetic metal ion to form a metal complex that is stable under physiological conditions. The metal chelator may be any of the metal chelators known in the art for complexing a medically useful paramagnetic metal ion, or radionuclide.

In some cases, such as in the case of complexes designed for radiopharmaceutical or radiotherapy applications, it can be convenient to prepare the complexes comprising a radionuclide, at or near the site where they are to be used (e.g., in a hospital pharmacy or clinic). Accordingly, in some embodiments, the complex can comprise a metal chelator uncomplexed with a metal ion. In such embodiments, the complex can be complexed with a suitable metal ion prior to administration. In other embodiments, the complex comprises a metal chelator complexed with a suitable metal ion (e.g., a paramagnetic metal ion or a radionuclide).

Suitable metal chelators include, for example, linear, macrocyclic, terpyridine, and N₃S, N₂S₂, or N₄ chelators (see also, U.S. Pat. Nos. 4,647,447, 4,957,939, 4,963,344, 5,367,080, 5,364,613, 5,021,556, 5,075,099, 5,886,142, the disclosures of which are incorporated by reference herein in their entirety), and other chelators known in the art including, but not limited to, HYNIC, DTPA, EDTA, DOTA, TETA, and bisamino bisthiol (BAT) chelators (see also U.S. Pat. No. 5,720,934). For example, macrocyclic chelators, and in particular N₄ chelators are described in U.S. Pat. Nos. 4,885,363; 5,846,519; 5,474,756; 6,143,274; 6,093,382; 5,608,110; 5,665,329; 5,656,254; and 5,688,487, the disclosures of which are incorporated by reference herein in their entirety. Certain N₃S chelators are described in PCT/CA94/00395, PCT/CA94/00479, PCT/CA95/00249 and in U.S. Pat. Nos. 5,662,885; 5,976,495; and 5,780,006, the disclosures of which are incorporated by reference herein in their entirety. The chelator may also include derivatives of the chelating ligand mercapto-acetyl-glycyl-glycyl-glycine (MAG3), which contains an N₃S, and N₂S2 systems such as MAMA (monoamidemonoaminedithiols), DADS (N₂S diaminedithiols), CODADS and the like. These ligand systems and a variety of others are described in Liu and Edwards, Chem. Rev. 1999, 99, 2235-2268; Caravan et al., Chem. Rev. 1999, 99, 2293-2352; and references therein, the disclosures of which are incorporated by reference herein in their entirety.

The metal chelator may also include complexes known as boronic acid adducts of technetium and rhenium dioximes, such as those described in U.S. Pat. Nos. 5,183,653; 5,387,409; and 5,118,797, the disclosures of which are incorporated by reference herein, in their entirety.

Examples of suitable chelators include, but are not limited to, derivatives of diethylenetriamine pentaacetic acid (DTPA), 1,4,7,10-tetraazacyclotetradecane-1,4,7,10-tetraacetic acid (DOTA), 1-substituted 1,4,7-tricarboxymethyl 1,4,7,10 tetraazacyclododecane triacetic acid (DO3A), derivatives of the 1-1-(1-carboxy-3-(p-nitrophenyl)propyl-1,4,7,10 tetraazacyclododecane triacetate (PA-DOTA) and MeO-DOTA, ethylenediaminetetraacetic acid (EDTA), and 1,4,8,11-tetraazacyclotetradecane-1,4,8,11-tetraacetic acid (TETA), derivatives of 3,3,9,9-tetramethyl-4,8-diazaundecane-2,10-dione dioxime (PnAO); and derivatives of 3,3,9,9-tetramethyl-5-oxa-4,8-diazaundecane-2,10-dione dioxime (oxa PnAO). Additional chelating ligands are ethylenebis-(2-hydroxy-phenylglycine) (EHPG), and derivatives thereof, including 5-Cl-EHPG, 5-Br-EHPG, 5-Me-EHPG, 5-t-Bu-EHPG, and 5-sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl-DTPA; bis-2 (hydroxybenzyl)-ethylene-diaminediacetic acid (HBED) and derivatives thereof; the class of macrocyclic compounds which contain at least 3 carbon atoms and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7,10-tetra(methyl tetraacetic acid), and benzo-TETMA, where TETMA is 1,4,8,11-tetraazacyclotetradecane-1,4,8,11-(methyl tetraacetic acid); derivatives of 1,3-propylenediaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris(2,3-dihydroxybenzoyl)aminomethylbenzene (MECAM). Examples of representative chelators and chelating groups are described in WO 98/18496, WO 86/06605, WO 91/03200, WO 95/28179, WO 96/23526, WO 97/36619, PCT/US98/01473, PCT/US98/20182, and U.S. Pat. Nos. 4,899,755, 5,474,756, 5,846,519 and 6,143,274, each of which is hereby incorporated by reference in its entirety.

In some embodiments, the metal chelator comprises desferrioxamine (also referred to as deferoxamine, desferrioxamine B, desferoxamine B, DFO-B, DFOA, DFB or desferal) or a derivative thereof. See, for example U.S. Pat. Nos. 8,309,583, 4,684,482, and 5,268,165, each of which is hereby incorporated by reference in its entirety for its teaching of desferrioxamine and desferrioxamine derivatives.

As is well known in the art, metal chelators can be specific for particular metal ions. Suitable metal chelators can be selected for incorporation into the self-assembling molecule based on the desired metal ion and intended use of the self-assembling molecule.

Paramagnetic ions form a magnetic moment upon the application of an external magnetic field thereto. Magnetization is not retained in the absence of an externally applied magnetic field because thermal motion causes the spin of unpaired electrons to become randomly oriented in the absence of an external magnetic field. By taking advantage of its property of shortening the magnetic relaxation time of water molecules, a paramagnetic substance is usable as an active component of MRI contrast agents. Suitable paramagnetic transition metal ions include Cr³⁺, Co²⁺, Mn²⁺, Ni²⁺, Fe²⁺, Fe³⁺, Zr⁴⁺, Cu²⁺, and Cu³⁺. In preferred embodiments, the paramagnetic ion is a lanthanide ion (e.g., La³⁺, Gd³⁺, Ce³⁺, Tb³⁺, Pr³⁺, Dy³⁺, Nd³⁺, Ho³⁺, Pm³⁺, Er³⁺, Sm³⁺, Tm³⁺, Eu³⁺, Yb³⁺, or Lu³⁺). In MRI, especially preferred metal ions are Gd³⁺, Mn²⁺, Fe³⁺, and Eu²⁺.

MRI contrast agents can also be made with paramagnetic nitroxides molecules in place of the chelating agent and paramagnetic metal ion.

Suitable radionuclides include ^{99m}Tc, ⁶⁷Ga, ⁶⁸Ga, ⁶⁶Ga, ⁴⁷Sc, ⁵¹Cr, ¹⁶⁷Tm, ¹⁴¹Ce, ¹¹¹In, ¹²³I, ¹²⁵I, ¹³¹I, ¹²⁴I, ¹⁸F, ¹¹C, ¹⁵N, 17O, ¹⁶⁸Y, ¹⁷⁵Y, ¹⁴⁰La, ⁹⁰Y, ⁸⁸Y, ⁸⁶Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ²²⁵Ac, ²¹¹At, ¹⁰⁵Rh, ¹⁰⁹Pd, ^{117m}Sn, ¹⁴⁹Pm, ¹⁶¹Tb, ¹⁷⁷Lu, ¹⁹⁸Au, ⁹⁹Au, 89Zr, and oxides or nitrides thereof. The choice of isotope will be determined based on the desired therapeutic or diagnostic application. For example, for diagnostic purposes (e.g., to diagnose and monitor therapeutic progress in primary tumors and metastases), suitable radionuclides include ⁶⁴Cu, ⁶⁷Ga, ⁶⁸Ga, ⁶⁶Ga, ^{99m}Tc, and ¹¹¹In, ¹⁸F, ⁸⁹Zr, ¹²³I, ¹³¹I, ¹²⁴I, ¹⁷⁷Lu, ¹⁵N, ¹⁷O. For therapeutic purposes (e.g., to provide radiotherapy for primary tumors and metastasis related to cancers of the prostate, breast, lung, etc.), suitable radionuclides include ⁶⁴Cu, ⁹⁰Y, ¹⁰⁵Rh, ¹¹¹In, ¹³¹I, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, ¹⁹⁹Au, ¹³¹I, and ¹²⁵I, ²¹²Bi, ²¹¹At.

In the case of complexes designed to be imaged using PET, radionuclides with short half-lives such as carbon-11 (˜20 min), nitrogen-13 (˜10 min), oxygen-15 (˜2 min), fluorine-18 (˜110 min), or rubidum-82 (˜1.27 min) are often used. In certain embodiments when a non-metal radionuclide is employed, the therapeutic or diagnostic agent comprises a radiotracer covalently attached to the self-assembling molecule. By way of exemplification, suitable ¹⁸F-based radiotracers include ¹⁸F-fluordesoxyglucose (FDG), ¹⁸F-dopamine, ¹⁸F-L-DOPA, ¹⁸F-fluorcholine, ¹⁸F-fluormethylethylcholin, and ¹⁸P-fluordihydrotestosteron.

In the case of self-assembled molecules designed to be imaged using PET, radionuclides with long half-lives such as ¹²⁴I, or ⁸⁹Zr are also often used.

Fluorescent imaging has emerged with unique capabilities for molecular cancer imaging. Fluorophores emit energy throughout the visible spectrum; however, the best spectrum for in vivo imaging is in the near-infrared (NIR) region (650 nm-900 nm). Unlike the visible light spectrum (400-650 nm), in the NIR region, light scattering decreases and photo absorption by hemoglobin and water diminishes, leading to deeper tissue penetration of light. Furthermore, tissue auto-fluorescence is low in the NIR spectra, which allows for a high signal to noise ratio. There is a range of small molecule organic fluorophores with excitation and emission spectra in the NIR region. Some, such as indocyanine green (ICG) and cyanine derivatives Cy5.5 and Cy7, have been used in imaging for a relatively long time. Modern fluorophores are developed by various biotechnology companies and include: Alexa dyes; IRDye dyes; VivoTag dyes and HylitePlus dyes. In general, the molecular weights of these fluorophores are below 1 kDa.

In some embodiments, the diagnostic agent can comprise a radiocontrast agent. In these embodiments, the diagnostic agent can comprise an iodinated moiety. Examples of suitable radiocontrast agents include iohexol, iodixanol and ioversol.

In some embodiments, the active agent can comprise a therapeutic agent. Any suitable therapeutic agent can be incorporated in the complexes described herein. In some examples, the therapeutic agent can comprise an agent to treat or prevent a disease or disorder associated with the overexpression of CD163. For example, in some cases, the therapeutic agent can comprise an anti-cancer agent, an anti-inflammatory agent, an agent that treats or prevents infection, or a combination thereof. In some examples, the active agent can comprise an agent administered to treat hemolytic anemia and other conditions characterized by or associated with hemolysis (e.g., sickle cell anemia, malaria, red blood cell transfusions, thalassemia, autoimmune disorders, bone marrow failure, infections, surgery, severe burns, acute lung injury, the administration of chemotherapeutics, radiation therapy, etc.). In some examples, the active agent can comprise an active agent administered to treat a disease associated with macrophages and monocytes. Such diseases are known in the art and include, for example, heart disease, HIV infection, cancer, fibrotic diseases (e.g., cystic fibrosis), asthma, inflammatory bowel disease, rheumatoid arthritis, and diseases in which macrophages or monocytes function as hosts for intracellular pathogens (e.g., malaria, tuberculosis, leishmaniasis, chikungunya, adenovirus, Legionnaires' disease, and infections caused by bacteria in the genus Brucella such as B. abortus, B. canis, B. melitensis, and B. suis).

In some embodiments, the active agent can comprise an anti-cancer agent. Examples of anti-cancer agents include, but are not limited to, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and I 131 Iodine Tositumomab), Bicalutamide, Bleomycin, Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Casodex (Bicalutamide), CeeNU (Lomustine), Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cytarabine, Cytarabine (Liposomal), Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folfox, Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine (Recombinant), HPV Quadrivalent Vaccine (Recombinant), Hycamtin (Topotecan Hydrochloride), Hyper-CVAD, Ibritumomab Tiuxetan, Ibrutinib, ICE, Iclusig (Ponatinib Hydrochloride), Ifex (Ifosfamide), lfosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R—CHOP, R—CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), Stanford V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and I¹³¹ Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), and Zytiga (Abiraterone Acetate). These anti-cancer agents are non-limiting, as the skilled artisan would be able to readily identify other anti-cancer agents.

In some embodiments, the active agent can comprise an anti-proliferative agent, e.g., mycophenolate mofetil (MMF), azathioprine, sirolimus, tacrolimus, paclitaxel, biolimus A9, novolimus, myolimus, zotarolimus, everolimus, or tranilast. These anti-proliferative agents are non-limiting, as the skilled artisan would be able to readily identify other anti-proliferative agents.

In some embodiments, the active agent can comprise an anti-inflammatory agent, e.g., corticosteroid anti-inflammatory drugs (e.g., beclomethasone, beclometasone, budesonide, flunisolide, fluticasone propionate, triamcinolone, methylprednisolone, prednisolone, or prednisone); or non-steroidal anti-inflammatory drugs (NSAIDs) (e.g., acetylsalicylic acid, diflunisal, salsalate, choline magnesium trisalicylate, ibuprofen, dexibuprofen, naproxen, fenoprofen, ketoprofen, dexketoprofen, fluribiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone, H-harpaide, or lysine clonixinate). These anti-inflammatory agents are non-limiting, as the skilled artisan would be able to readily identify other anti-inflammatory agents.

In some embodiments, the active agent can comprise a drug that prevents or reduces transplant rejection, e.g., an immunosuppressant. Exemplary immunosuppressants include calcineurin inhibitors (e.g., cyclosporine, Tacrolimus (FK506)); mammalian target of rapamycin (mTOR) inhibitors (e.g., rapamycin, also known as Sirolimus); antiproliferative agents (e.g., azathioprine, mycophenolate mofetil, mycophenolate sodium); antibodies (e.g., basiliximab, daclizumab, muromonab); corticosteroids (e.g., prednisone). These drugs that prevent or reduce transplant rejection are non-limiting, as the skilled artisan would be able to readily identify other drugs that prevent or reduce transplant rejection.

In some embodiments, the active agent can comprise a drug that treats or prevents infection, e.g., an antibiotic. Suitable antibiotics include, but are not limited to, beta-lactam antibiotics (e.g., penicillins, cephalosporins, carbapenems), polymyxins, rifamycins, lipiarmycins, quinolones, sulfonamides, macrolides lincosamides, tetracyclines, aminoglycosides, cyclic lipopeptides (e.g., daptomycin), glycylcyclines (e.g., tigecycline), oxazonidinones (e.g., linezolid), and lipiarmycines (e.g., fidazomicin). For example, antibiotics include erythromycin, clindamycin, gentamycin, tetracycline, meclocycline, (sodium) sulfacetamide, benzoyl peroxide, and azelaic acid. Suitable penicillins include amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, and ticarcillin. Exemplary cephalosporins include cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cfcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, ceftazidime, cefclidine, cefepime, ceflurprenam, cefoselis, cefozopran, cefpirome, cequinome, ceflobiprole, ceftaroline, cefaclomezine, cefaloram, cefaparole, cefcanel, cefedrlor, cefempidone, cefetrizole, cefivitril, cefmatilen, cefmepidium, cefovecin, cefoxazole, cefrotil, cefsumide, cefuracetime, and ceftioxide. Monobactams include aztreonam. Suitable carbapenems include imipenem/cilastatin, doripenem, meropenem, and ertapenem. Exemplary macrolides include azithromycin, erythromycin, larithromycin, dirithromycin, roxithromycin, and telithromycin. Lincosamides include clindamycin and lincomycin. Exemplary streptogramins include pristinamycin and quinupristin/dalfopristin. Suitable aminoglycoside antibiotics include amikacin, gentamycin, kanamycin, neomycin, netilmicin, paromomycin, streptomycin, and tobramycin. Exemplary quinolones include flumequine, nalidixic acid, oxolinic acid, piromidic acid, pipemidic acid, rosoxacin, ciprofloxacin, enoxacin, lomefloxacin, nadifloxacin, norfloxacin, ofoxacin, pefloxacin, rufloxacin, balofloxacin, gatifloxacin, repafloxacin, levofloxacin, moxifloxacin, pazufloxacin, sparfloxacin, temafloxacin, tosufloxacin, besifloxacin, clinafoxacin, gemifloxacin, sitafloxacin, trovafloxacin, and prulifloxacin. Suitable sulfonamides include sulfamethizole, sulfamethoxazole, and trimethoprim-sulfamethoxazone. Exemplary tetracyclines include demeclocycline, doxycycline, minocycline, oxytetracycline, tetracycline, and tigecycline. Other antibiotics include chloramphenicol, metronidazole, tinidazole, nitrofurantoin, vancomycin, teicoplanin, telavancin, linezolid, cycloserine, rifampin, rifabutin, rifapentin, bacitracin, polymyxin B, viomycin, and capreomycin. The skilled artisan could readily identify other antibiotics useful in the devices and methods described herein.

In some embodiments, the active agent can comprise an anti-HIV agent. Examples of anti-HIV agents include anti-HIV antibodies, immunostimulants such as interferon, and the like, a reverse transcriptase inhibitor, a protease inhibitor, an inhibitor of bond between a bond receptor (CD4, CXCR4, CCR5, and the like) of a host cell recognized by virus and the virus, and the like.

Specific examples of HIV reverse transcriptase inhibitors include Retrovir® (zidovudine or AZT), Epivir® (lamivudine or 3TC), Zerit® (sanilvudine), Videx® (didanosine), Hivid® (zalcitabine), Ziagen® (abacavir sulfate), Viramune® (nevirapine), Stocrin® (efavirenz), Rescriptor® (delavirdine mesylate), Combivir® (zidovudine+lamivudine), Trizivir® (abacavir sulfate+lamivudine+zidovudine), Coactinon® (emivirine), Phosphonovir®, Coviracil®, alovudine (3′-fluoro-3′-deoxythymidine), Thiovir (thiophosphonoformic acid), Capravirin (5-[(3,5-dichlorophenyl)thio]-4-isopropyl-1-(4-pyridylmethyl)imidazole-2-methanol carbamic acid), Tenofovir (PMPA), Tenofovir disoproxil fumarate ((R)-[[2-(6-amino-9H-purin-9-yl)-1-methylethoxy]methyl]phosphonic acid bis(isopropoxycarbonyloxymethyl)ester fumarate), DPC-083 ((4S)-6-chloro-4-[(1E)-cyclopropylethenyl]-3,4-dihydro-4-trifluoromethyl-2 (1H)-quinazolinone), DPC-961 ((4S)-6-chloro-4-(cyclopropylethynyl)-3,4-dihydro-4-(trifluoromethyl)-2 (1H)-quinazolinone), DAPD ((−)-β-D-2,6-diaminopurine dioxolane), Immunocal, MSK-055, MSA-254, MSH-143, NV-01, TMC-120, DPC-817, GS-7340, TMC-125, SPD-754, D-A4FC, capravirine, UC-781, emtricitabine, alovudine, Phosphazid, UC-781, BCH-10618, DPC-083, Etravirine, BCH-13520, MIV-210, abacavir sulfate/lamivudine, GS-7340, GW-5634, GW-695634, and the like.

Specific examples of HIV protease inhibitors include Crixivan® (indinavir sulfate ethanolate), saquinavir, Invirase® (saquinavir mesylate), Norvir® (ritonavir), Viracept® (nelfinavir mesylate), lopinavir, Prozei® (amprenavir), Kaletra® (ritonavir+lopinavir), mozenavir dimesylate ([4R-(4α,5α,6β)]-1-3-bis[(3-aminophenyl)methyl]hexahydro-5,6-dihydroxy-4,7-bis(phenylmethyl)-2H-1,3-diazepin-2-one dimethanesulfonate), tipranavir (3′-[(1R)-1-[(6R)-5,6-dihydro-4-hydroxy-2-oxo-6-phenylethyl-6-propyl-2H-pyran-3-yl]propyl]-5-(trifluoromethyl)-2-pyridinesulfonamide), lasinavir (N-[5(S)-(tert-butoxycarbonylamino)-4(S)-hydroxy-6-phenyl-2(R)-(2,3,4-trimethoxybenzyl)hexanoyl]-L-valine 2-methoxyethylenamide), KNI-272 ((R)—N-tert-butyl-3-[(2S,3S)-2-hydroxy-3-N—[(R)-2-N-(isoquinolin-5-yloxyacetyl)amino-3-methylthiopropanoyl]amino-4-phenylbutanoyl]-5,5-dimethyl-1,3-thiazolidine-4-carboxamide), GW-433908, TMC-126, DPC-681, buckminsterfullerene, MK-944A (MK944 (N-(2(R)-hydroxy-1(S)-indanyl)-2(R)-phenylmethyl-4(S)-hydroxy-5-[4-(2-benzo[b]furanylmethyl)-2(S)-(tert-butylcarbamoyl)piperazin-1-yl]pentanamide)+indinavir sulfate), JE-2147 ([2(S)-oxo-4-phenylmethyl-3(S)-[(2-methyl-3-oxy)phenylcarbonylamino]-1-oxabutyl]-4-[(2-methylphenyl)methylamino]carbonyl-4(R)-5,5-dimethyl-1,3-thiazole), BMS-232632 ((3S,8S,9S,12S)-3,12-bis(1,1-dimethylethyl)-8-hydroxy-4,11-dioxo-9-(phenylmethyl)-6-[[4-(2-pyridinyl)phenyl]methyl]-2,5,6,10,13-pentaazatetradecanedicarboxylic acid dimethyl ester), DMP-850 ((4R,5S,6S,7R)-1-(3-amino-1H-indazol-5-ylmethyl)-4,7-dibenzyl-3-butyl-5,6-dihydroxyperhydro-1,3-diazepin-2-one), DMP-851, RO-0334649, Nar-DG-35, R-944, VX-385, TMC-114, Tipranavir, Fosamprenavir sodium, Fosamprenavir calcium, Darunavir, GW-0385, R-944, RO-033-4649, AG-1859, and the like.

The HIV integrase inhibitor may be S-1360, L-870810, and the like. The DNA polymerase inhibitor or DNA synthesis inhibitor may be Foscavir®, ACH-126443 (L-2′,3′-didehydro-dideoxy-5-fluorocytidine), entecavir ((1S,3S,4S)-9-[4-hydroxy-3-(hydroxymethyl)-2-methylenecyclopentyl]guanine), calanolideA([10R-(10α,11β,12α)]-11,12-dihydro-12-hydroxy-6,6,10,11-tetramethyl-4-propyl-2H,6H,10H-benzo[1,2-b:3,4-b′.5,6-b″ ]tripyran-2-one), calanolide B, NSC-674447 (1,1′-azobisformamide), Iscador (viscum alubm extract), Rubutecan, and the like. The HIV antisense drug may be HGTV-43, GEM-92, and the like. The anti-HIV antibody or other antibody may be NM-01, PRO-367, KD-247, Cytolin®, TNX-355 (CD4 antibody), AGT-1, PRO-140 (CCR5 antibody), Anti-CTLA-4 Mab, and the like. The HIV vaccine or other vaccine may be ALVAC®, AIDSVAX®, Remune®, HIV gp41 vaccine, HIV gp120 vaccine, HIV gp140 vaccine, HIV gp160 vaccine, HIV p17 vaccine, HIV p24 vaccine, HIV p55 vaccine, AlphaVax Vector System, canarypox gp160 vaccine, AntiTat, MVA-F6 Nef vaccine, HIV rev vaccine, C4-V3 peptide, p2249f, VIR-201, HGP-30W, TBC-3B, PARTICLE-3B, and the like, Antiferon (interferon-α vaccine), and the like.

The interferon or interferon agonist may be Sumiferon®, MultiFeron®, interferon-τ, Reticulose, Human leukocyte interferon alpha, and the like. The CCR5 antagonist may be SCH-351125, and the like. The pharmaceutical agent acting on HIV p24 may be GPG-NH2 (glycyl-prolyl-glycinamide), and the like. The HIV fusion inhibitor may be FP-21399 (1,4-bis[3-[(2,4-dichlorophenyl)carbonylamino]-2-oxo-5,8-disodium sulfonyl]naphthyl-2,5-dimethoxyphenyl-1,4-dihydrazone), T-1249, Synthetic Polymeric Construction No 3, pentafuside, FP-21399, PRO-542, Enfuvirtide, and the like. The IL-2 agonist or antagonist may be interleukin-2, Imunace®, Proleukin®, Multikine®, Ontak®, and the like. The TNF-α antagonist may be Thalomid® (thalidomide), Remicade® (infliximab), curdlan sulfate, and the like. The α-glucosidase inhibitor may be Bucast®, and the like.

The purine nucleoside phosphorylase inhibitor may be peldesine (2-amino-4-oxo-3H,5H-7-[(3-pyridyl)methyl]pyrrolo[3,2-d]pyrimidine), and the like. The apoptosis agonist or inhibitor may be Arkin Z®, Panavir®, Coenzyme Q10 (2-deca(3-methyl-2-butenylene)-5,6-dimethoxy-3-methyl-p-benzoquinone), and the like. The cholinesterase inhibitor may be Cognex®, and the like, and the immunomodulator may be Imunox®, Prokine®, Met-enkephalin (6-de-L-arginine-7-de-L-arginine-8-de-L-valinamide-adrenorphin), WF-10 (10-fold dilute tetrachlorodecaoxide solution), Perthon, PRO-542, SCH-D, UK-427857, AMD-070, AK-602, and the like.

In addition, Neurotropin®, Lidakol®, Ancer 20®, Ampligen®, Anticort®, Inactivin®, and the like, PRO-2000, Rev M10 gene, HIV specific cytotoxic T cell (CTL immunotherapy, ACTG protocol 080 therapy, CD4-(gene therapy), SCA binding protein, RBC-CD4 complex, Motexafin gadolinium, GEM-92, CNI-1493, (±)-FTC, Ushercell, D2S, BufferGel®, VivaGel®, Glyminox vaginal gel, sodium lauryl sulfate, 2F5, 2F5/2G12, VRX-496, Ad5gag2, BG-777, IGIV-C, BILR-255, and the like may be used in the combination therapy.

Other suitable active agents include porphyrin-based active agents (e.g., porphyrin-based imaging agents, porphyrin-based agents for photodynamic therapy), erythropoietin, hydroxycarbamide (also known as hydroxyurea), corticosteroids, immunosuppressive agents, analgesic agents, agents that induce hemolysis (e.g., rituximab, cephalosporins, dapsone, levodopa, levofloxacin, methyldopa, nitrofurantoin, NSAIDs, penicillin and derivatives thereof, phenazopyridine, quinidine), dexamethasone, conjugates targeting the CD163 receptor (e.g., agents described in U.S. Pat. No. 9,724,426 to Graversen et al. which is incorporated by reference in its entirety), antibiotics; anti-tuberculosis antibiotics (such as isoniazide, ethambutol); anti-retroviral drugs, for example inhibitors of reverse transcription (such as zidovudin) and/or protease inhibitors (such as indinavir); drugs with effect on leishmaniasis (such as meglumine antimoniate); immunosuppressive drugs such as a glucocorticoid (e.g., cortisone and derivatives thereof (such as hydrocortisone); prednisone and derivatives thereof (such as prednisolone, methylprednisolone, methylprednisolone-acetate, methylprednisolone-succinate); dexamethasone and derivatives thereof; triamcinolone and derivatives thereof (such as triamcinolonehexacetonuid, triamcinolonacetonamid); paramethasone; betamethasone; fluhydrocortisone; fluocinolone); methotrexate; cyclophosphamide; 6-mercaptopurin; cyclosporine; tacrolimus; mycophenolate mofetil; sirulimus; everolimus; an siRNA molecule capable of inhibiting synthesis of proinflammatory cytokines (such as TNF); a non-steroidal anti-inflammatory drug (NSAIDs, such as aspirin, ibuprofen); a steroid (such as vitamin D); and a disease-modifying anti-rheumatic drug (DMARDs, such as penicillamin, sulfasalazin, cyclosporine).

In some embodiments, the active agent can comprise a toll-like receptor (TLR) agonist. A “TLR agonist” as used herein, refers to a substance that can combine with a TLR and activate it. By slightly altering the structure of such substances, TLR agonists can be designed to have different stabilities in the body, allowing a certain amount of control over where the substances go, and how long they last. Microbial ligands have been identified for several mammalian TLRs. For example, TLR4 recognizes lipopolysaccharide (LPS), TLR2 interacts with peptidoglycan, bacterial lipopeptides, and certain types of LPS, TLR3 recognizes double-stranded RNA, TLR5 recognizes bacterial flagellin, TLR9 recognizes bacterial DNA.

TLR agonists are well-known in the art and include, for example, but not limited to, lipopolysaccharide (LPS, binds TLR4), Fibrin (binds TLR4), lipoteichoic acid (LTA, binds TLR2), peptidoglycan (PG, binds TLR2), CpG (bacterial DNA, binds TLR9), 7-thia-8-oxoguanosine (TOG or isatoribine, binds TLR7), 7-deazaguanosine (binds TLR7), 7-allyl-8-oxoguanosine (loxoribine, binds TLR7), 7-dezaguanosine (7-deza-G, binds TLR7), imiquimod (R837, binds TLR7), or R848 (binds TLR7). In some embodiments, the TLR agonist can comprise a TLR7 agonist or a TLR9 agonist that is carried by the apoHb-Hp complex for receptor mediated uptake and immune activation within the endosome. In certain embodiments, the the TLR agonist can comprise a TLR7 agonist (e.g., an imidazoquinoline such as imiquimod).

The expression of heme oxygenase-1 (HO-1) inhibits vascular inflammation and the induction of apoptosis. Accordingly, in some embodiments, the active agent can comprise an agent which modulates the activity of heme-oxygenase-1 (HO-1) activity. In some embodiments, the modulator of HO-1 is an antagonist, partial agonist, inverse agonist, neutral or competitive antagonist, allosteric antagonist, and/or orthosteric antagonist of HO-1. In some embodiments, the modulator of HO-1 is a HO-1 agonist, partial agonist, and/or positive allosteric modulator. In some embodiments, the agonist, partial agonist, and/or positive allosteric modulator of HO-1 is piperine, hemin, and/or brazilin. In some embodiments, the active agent comprises a protoporphyrin IX complex, such as zinc protoporphyrin IX or tin protoporphyrin IX, that is a HO-1 antagonist.

In some embodiments, pharmaceutical compositions methemoglobin or polymerized methemoglobin prepared using the methods described herein can be co-administered with haptoglobin, hemopexin, transferrin or human serum albumin.

These pharmaceutical compositions can be administered to a subject in need thereof to treat cyanide or hydrogen sulfide poisoning.). Compositions comprising hydrogen sulfide bound to the methemoglobin, the polymerized methemoglobin, or the methemoglobin complexed with haptoglobin can also be used to deliver a subtoxic dose of hydrogen sulfide to lower the metabolism of a cell, tissue, organ, or subject.

The compositions described herein can also be used to target the delivery of drugs to macrophages or monocytes (e.g., to down-regulate production of inflammatory cytokines, to kill intracellular organisms, or to kill malignant cells). In this way, the compositions can be used to selectively deliver active agents that significant impact certain diseases while minimizing adverse impacts of the active agent on other cells in the body.

In some embodiments, the compositions can be administered to a subject in need thereof to treat a disease characterized by the overexpression of CD163. Such diseases are known in the art, and include but not limited to, for example cancer (e.g., breast cancer, Hodgkin Lymphoma), liver cirrhosis, type 2 diabetes, macrophage activation syndrome, Gaucher's disease, sepsis, HIV infection, and rheumatoid arthritis.

In some embodiments, the compositions can be administered to a subject in need thereof to treat a disease which involves macrophages or monocytes. Such diseases are known in the art and include, for example, heart disease, HIV infection, cancer, fibrotic diseases (e.g., cystic fibrosis), asthma, inflammatory bowel disease, rheumatoid arthritis, and diseases in which macrophages or monocytes function as hosts for intracellular pathogens (e.g., malaria, tuberculosis, leishmaniasis, chikungunya, adenovirus, Legionnaires' disease, coronavirus (e.g., SARS-CoV-2, SARS, MERS, etc.), and infections caused by bacteria in the genus Brucella such as B. abortus, B. canis, B. melitensis, and B. suis).

The compositions may also be used to triger CD163+ uptake of drug-conjugated Hp. In one example, in addition to administering the full methemoglobin-Hp-drug complex, only the Hp-drug conjugate or methemoglobin-drug conjugate could be administered. Then, methemoglobin or Hp could be administered to induce macrophage and monocyte uptake of the complex. In doing so, the Hp-drug conjugate would have a longer circulatory half-life to perform its desired function. For example, similar to bispecific monoclonal antibodies, Hp could be complexed with a targeting agent and macrophage/monocyte uptake could be trigged by the injection of methemoglobin. Such a therapeutic approach could be employed to treat cancer. For cancer treatment, Hp conjugated with a cancer cell targeting molecule could be administered to a patient. The Hp conjugated with the cancer cell targeting molecule would circulate until it binds to the surface of the cancer cell. Subsequent administration of methemoglobin would bind to the Hp attached to the cancer cell. The resulting methemoglobin-Hp complex attached to the cancer cell would recruit macrophages and monocytes for phagocytosis of the cancerous cell.

In some embodiments, methods can further include administering an agent to a patient to modulate CD163 expression (and by extension circulation and/or delivery of the complexes described herein). For example, methods can comprise administering a gluticosteroid to the patient to increase expression of CD163 or administering an agent (e.g., a gene silencing agent) to decrease expression of CD163.

In some embodiments, the methemoglobin-Hp complex and an active agent coordinated thereto is administered in combination with an immunotherapy agent, such as an immune checkpoint inhibitor. In some examples, the immune checkpoint inhibitor can comprise an anti-PD1 or anti-PDL1 antibody. In some examples, the immune checkpoint inhibitor can comprise an anti-CTLA4 monoclonal antibody.

In some embodiments, the disease can involve cellular iron accumulation and ferroptosis. In some embodiments, the methemoglobin-Hp complex and the active agent (e.g., HO-1 enzyme agonist) coordinated thereto are administered in combination with a ferropototic agent, such as Bay117085 or withaferin A.

Also provided are populations of particles comprising hemoglobin or polymerized methemoglobin prepared using the methods described herein. In some embodmiments, the methemoglobin can be complexed with haptoglobin.

In some embodiments, the particles can comprise nanoparticles. The term “nanoparticle,” as used herein, generally refers to a particle of any shape having one or more dimensions ranging from 1 nm up to, but not including, 1 micron. In some embodiments, the nanoparticles can comprise a particle of any shape having one or more dimensions ranging from 1 nm up to, but not including, 1 micron; and one or more dimensions of 1 micron or more (e.g., from 1 micron to 10 microns, from 1 micron to 20 microns, from 1 micron to 25 microns, from 1 micron to 50 microns, from 1 micron to 100 microns, or from 1 micron to 150 microns).

In other embodiments, the particles can comprise microparticles. The microparticles can be of any shape, and have one or more dimensions ranging from 1 micron to 150 microns (e.g., from 1 micron to 100 microns, or from 1 micron to 50 microns). In some embodiments, all dimensions can range from 1 micron to 150 microns (e.g., from 1 micron to 100 microns, or from 1 micron to 50 microns).

In other embodiments, the particles can be macroscopic (e.g., they can have an average particle size of up to 1 cm, or greater).

In some embodiments, the particles can have an average particle size of less than 1 cm (e.g., less than 1000 microns, less than 750 microns, less than 500 microns, less than 250 microns, less than 200 microns, less than 150 microns, less than 100 microns, less than 50 microns, less than 25 microns, less than 20 microns, less than 10 microns, less than 5 microns, less than 1 micron, less than 750 nm, less than 500 nm, less than 250 nm, less than 200 nm, less than 150 nm, less than 100 nm, less than 50 nm, or less than 25 nm). In some embodiments, some embodiments, the particles can have an average particle size of at least 10 nm (e.g., at least 25 nm, at least 50 nm, at least 100 nm, at least 150 nm, at least 200 nm, at least 250 nm, at least 500 nm, at least 750 nm, at least 1 micron, at least 5 microns, at least 10 microns, at least 20 microns, at least 25 microns, at least 50 microns, at least 100 microns, at least 150 microns, at least 200 microns, at least 250 microns, at least 500 microns, at least 750 microns, or at least 1000 microns). The particles can have an average particle size ranging from any of the minimum values described above to any of the maximum values described above. For example, in certain embodiments, the particles can have an average particle size of from 10 nm to 1 cm (e.g., from 10 nm to 1 micron). The term “average particle size,” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a nonspherical particle may refer to the largest linear distance between two points on the surface of the particle. Mean particle size can be measured using methods known in the art, such as dynamic light scattering or electron microscopy.

In some embodiments, the particles comprise a population of nanoparticles having a monodisperse particle size distribution. The term “monodisperse,” as used herein, describes a population of particles where all of the particles are the same or nearly the same size. As used herein, a monodisperse particle size distribution refers to particle distributions in which 80% of the distribution (e.g., 85% of the distribution, 90% of the distribution, or 95% of the distribution) lies within 20% of the median particle size (e.g., within 15% of the median particle size, within 10% of the median particle size, or within 5% of the median particle size).

In other embodiments, the population of particles are a polydisperse population of particles. In some instances where the population of particles is polydisperse, greater that 50% of the particle size distribution, more preferably 60% of the particle size distribution, most preferably 75% of the particle size distribution lies within 10% of the median particle size.

Methemoglobin or polymerized methemoglobin prepared using the methods described herein can also be used to scavenge NO₂⁻ ions (e.g., in vivo or in any ex vivo environment). Methemoglobin or polymerized methemoglobin prepared using the methods described herein can also be used to scavenge cyanide or hydrogen sulfide ex vivo (e.g., in wastewater samples, water used in mining/refining operations, or other environmental remediation applications where cyanide or hydrogen sulfide is a contaminant). The particulate compositions described above can be particularly suitable for these purposes, for example, when used as a stationary phase, in a filter, filter bed, or separation column to remove or scavenge NO₂⁻ ions, cyanide, and/or hydrogen sulfide.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: Scalable Manufacturing Platform for the Production of Methemoglobin as a Non-Oxygen Carrying Control Material

### SUMMARY

Methemoglobin (metHb) arises from the oxidation of ferrous hemoglobin (HbFe²⁺, Hb) to ferric hemoglobin (HbFe³⁺, metHb), which is unable to bind gaseous ligands such as oxygen (O₂), nitric oxide (NO) and carbon monoxide (CO). Therefore, metHb does not elicit vasoconstriction and systemic hypertension in vivo due to NO scavenging in comparison to cell-free Hb, but will induce oxidative tissue injury, demonstrating the potential of using metHb as a control material when studying the toxicity of cell-free Hb. Hence, the goal of this work was to develop a novel manufacturing strategy for production of metHb that is amenable to scale-up. In this example, small scale (i.e., 1 mL reaction volume) screening experiments were initially conducted to determine the optimal molar ratio of Hb to the oxidization agents hydrogen peroxide (H₂O₂) or sodium nitrite (NaNO₂) to achieve the highest conversion of Hb into metHb. During the course of this study, a spectral deconvolution program was employed to determine the molar fraction of various species (hemichrome, metHb, oxyHb, metHb-NO₂⁻, and NaNO₂) in solution during the oxidation reaction. From this analysis, either a 1:1 or 1:5 molar ratio was identified as optimal molar ratios of Hb:NaNO₂ (heme basis) that yielded the highest conversion of Hb into metHb with negligible amounts of side products. Hence in order to reduce the reaction time, a 1:5 molar ratio was chosen for large scale (i.e., 1.5 L reaction volume) metHb synthesis, which achieved yields >75% with purities >85%. The biophysical properties of metHb was then characterized to elucidate the potential of using the synthesized metHb as a non-O₂ carrying control material. The haptoglobin binding kinetics of metHb was found to be similar to Hb. Additionally, the synthesized metHb was stable in phosphate buffered saline (50 mM, pH 7.4) at 4° C. for approximate one week, indicating the high stability of the material.

### BACKGROUND

In this example, to synthesize pure metHb, either sodium nitrite (NaNO₂) or hydrogen peroxide (H₂O₂) was used to oxidize Hb. Hence, various molar ratios of Hb:oxidization agent were screened in small scale reaction volumes (1 mL) to determine the optimal type of oxidization agent and molar ratio of Hb:oxidization agent that yielded the highest conversion of Hb into metHb. To monitor the oxidation of Hb during the reaction, a spectral deconvolution program (Alchromy) was employed to determine the molar fraction of various species (hemichrome, metHb, oxyHb, metHb-NO₂⁻, and NaNO₂) in solution based on the standard UV-visible spectra of each pure species. From this analysis, we identified an optimal molar ratio of Hb:NaNO₂ that yielded the highest conversion of Hb into metHb with negligible levels of impurities.

To scale up production of metHb, a synthesis and purification protocol was developed. In the metHb synthesis protocol, NaNO₂ was injected into a well-mixed batch reactor containing bovine Hb (bHb) or human Hb (hHb). After the reaction went to completion, the synthesized metHb was then subject to diafiltration with LR solution until ˜250 mL of the product was obtained at a total protein concentration of ˜100 mg/mL and >85% metHb level. The biophysical properties of metHb were then characterized to elucidate the potential of using it as a non-O₂ carrying control material. At the end of the tangential flow filtration (TFF) purification process, only metHb was present in solution with undetectable levels of nitrite. Each batch of metHb was characterized via spectral deconvolution analysis, Hp binding kinetics analysis, size exclusion HPLC (SEC-HPLC) analysis, and subjected to storage stability analysis.

### Materials and Methods

Materials. Sodium citrate anticoagulated whole bovine blood was purchased from Quad Five (Ryegate, MT). Expired packed human red blood cell (RBC) units were obtained from Transfusion Services (Wexner Medical Center, The Ohio State University, Columbus, Ohio). Sodium nitrite (NaNO₂), hydrogen peroxide (H₂O₂), calcium chloride (CaCl₂)₂·H₂O), sodium phosphate monobasic (NaH₂PO₄), sodium phosphate dibasic (Na₂HPO₄), potassium chloride (KCl), sodium hydroxide (NaOH), sodium chloride (NaCl), and sodium lactate were purchased from Sigma-Aldrich (St. Louis, MO). Hollow fiber (HF) modules with 500 kDa and 50 kDa molecular weight cut off (MWCO) were procured from Repligen (Rancho Dominguez, CA). Potassium ferricyanide (K₃Fe(CN)₆) and potassium cyanide (KCN) were obtained from Fisher Scientific (Pittsburgh, PA). 0.2 μm syringe filters were purchased from Thermo Fisher Scientific (Waltham, MA).

Hb Purification. Bovine/human red blood cells (RBCs) were washed via centrifugation with 0.9% saline and lysed with phosphate buffer (PB) (3.75 mM, pH 7.4). Tangential flow filtration (TFF) HF modules with MWCOs of 500 kDa and 50 kDa were then used to purify and concentrate bHb/hHb as described in the literature (Palmer, Sun, & Harris, 2009).

Small Scale Optimization of Hb:Oxidization Agent Stoichiometry. A small scale (1 mL) metHb synthesis study using two different types of oxidization agents was conducted to determine the optimal dose of oxidizing agent needed to fully convert Hb into metHb. In this study, hydrogen peroxide (H₂O₂) and sodium nitrite (NaNO₂) were prepared at 1:1, 1:5, and 1:10 molar ratio of Hb:oxidization agent within 1 mL UV-visible quartz cuvettes. bHb/hHb was diluted to 20 mg/mL using phosphate buffer (PBS, 0.1 M, pH 7.4). Stock solutions of NaNO₂ and H₂O₂ were prepared at 8.5 mg/mL and 4.2 mg/mL, respectively. To obtain the 1:1, 1:5, and 1:10 molar ratio of Hb:oxidization agent solution, 1 mL bHb/hHb solution was mixed with 10/50/100 μL of the NaNO₂/H₂O₂ stock solution, respectively. The absorbance spectra from 350-700 nm was simultaneously monitored for 21 hours at room temperature in 6 parafilm-sealed quartz cuvettes and a blank quartz cuvette with PBS (0.1 M, pH 7.4) using an HP 8452A diode array UV-visible spectrometer (Olis, Bogart, GA).

To determine the fraction of multiple Hb species in solution, we developed a spectral deconvolution program (Alchromy) based on the extinction coefficients and absorbance spectra of each pure species. For the reaction between bHb/hHb and NaNO₂, the fractional composition of hemichrome, oxyHb, metHb, ferrylHb, metHb-NO₂⁻, and NaNO₂ in solution was calculated at each time point during the 21-hour reaction time period using the Alchromy program. Similarly, for the reaction between bHb/hHb and H₂O₂, the fractional composition of hemichrome, oxyHb, and metHb was calculated as described above.

UV-Visible Spectra Deconvolution Analysis. Samples containing Hb were analyzed via UV-visible spectroscopy and compared to the UV-visible spectra of pure species of Hb bound to various ligands through spectral deconvolution. The open-source Python package Alchromy (www.alchromy.com) was used to interpret and analyze spectra, and leveraged a nonlinear least squares fitting function of the SciPy package to determine the fraction of various liganded forms of Hb that contribute to the final spectra of the Hb mixture (Pires et al., 2020).

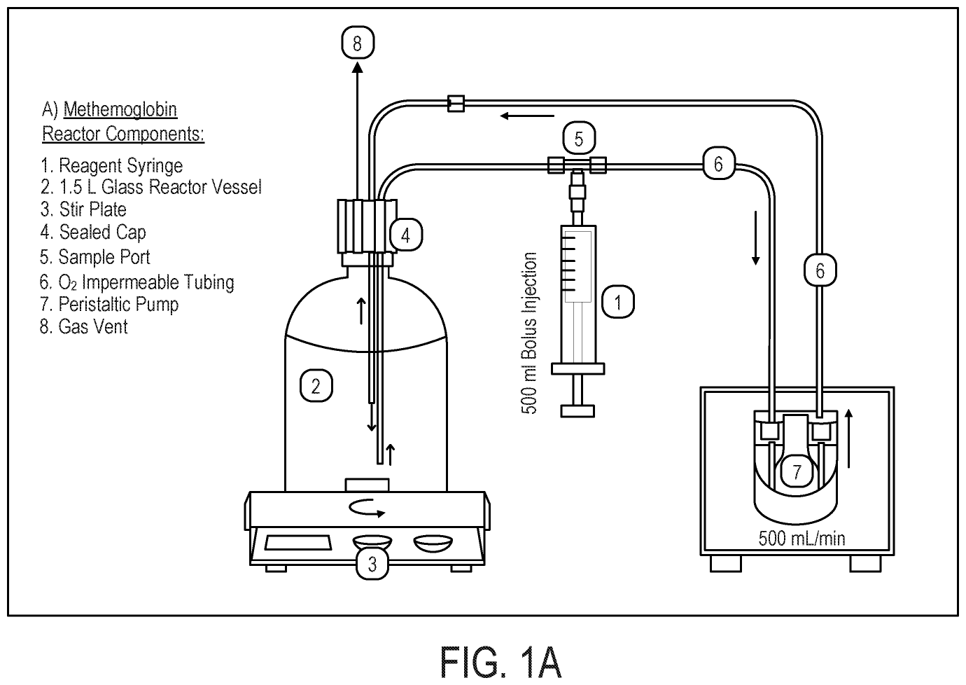

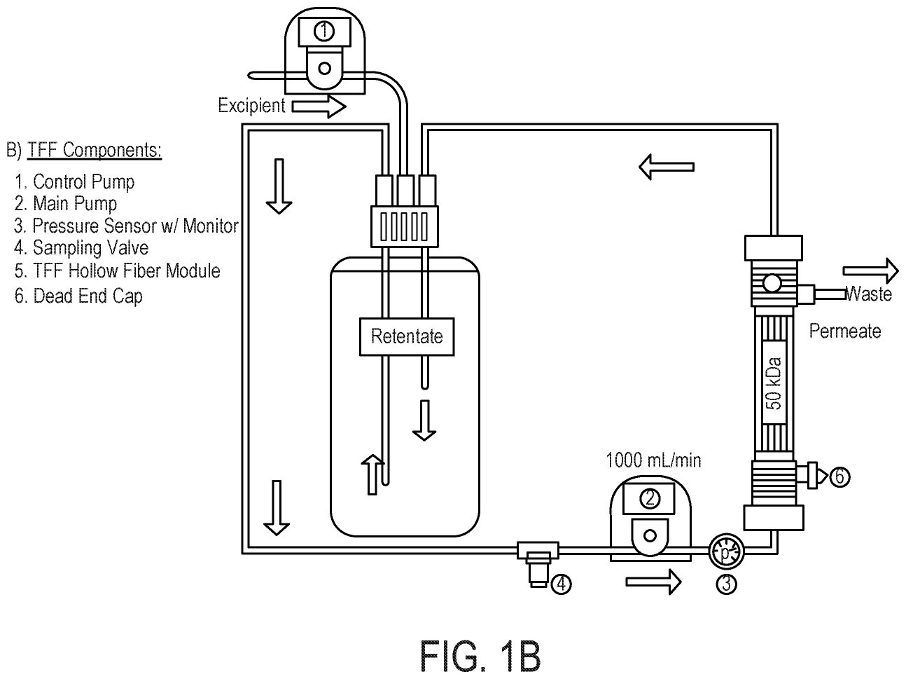

Large Scale MetHb Synthesis. Initially, 30 g of bHb/hHb was diluted in 1.5 L phosphate buffer saline (PBS, 0.1 M, pH 7.4) and placed into an airtight, amber-tinted reactor vessel with continuous stirring as shown in FIG. 1A. The reactor vessel coupled with a recirculation loop (500 m/min) was placed in a fume hood at room temperature. After the recirculation loop was turned on, a bolus injection of 50 mL NaNO₂ solution (0.013 mg/mL) was initiated through a 50 mL syringe attached to the sampling port at the inlet side of the reactor vessel. After two hours of reaction with constant stirring and recirculation, the bovine/human metHb (metbHb/methHb) solution was refrigerated at 4° C. overnight.

MetHb Clarification and Purification. As shown in FIG. 1B, both metbHb and methHb were initially transferred into a 2 L Nalgene bottle and concentrated down to 300 mL via TFF on a 50 kDa HF module. The metbHb/methHb solution was then transferred into a 1 L Nalgene bottle and subject to constant volume diafiltration with reductant-free modified lactated Ringer's solution (115 mM NaCl, 4 mM KCl, 1.4 mM CaCl₂·2H₂O, 13 mM NaOH, 27 mM sodium lactate, pH 7.4). After 6 diafiltration cycles, the final metbHb/methHb product was concentrated to 270 mL at ˜100 mg/mL and stored at −80° C.

### Characterization of MetHb

Multi-Species Analysis During TFF. To evaluate the composition of metbHb/methHb mixtures as a function of TFF processing time, samples were collected every hour during TFF processing. The fraction of multiple Hb species at each time point including hemichrome, oxyHb, metHb, ferrylHb, metHb-NO₂⁻, and NaNO₂ was determined by spectra deconvolution using the Alchromy program.

Haptoglobin-Binding Kinetics Study. To evaluate haptoglobin (Hp)-Hb/metHb binding kinetics, a Hp mixture (0.25 μM, Hb tetramer binding basis) containing a mixture of Hp2-1 and Hp2-2 was purified from human Cohn Fraction IV (Pires & Palmer, 2020). The kinetics of Hp binding to Hb/metHb was measured as previously described in the literature (Meng et al., 2018). The reaction between Hp and Hb/metHb was followed by stopped flow fluorescence spectrometry by excitation at 285 nm and monitoring the fluorescence emission at 310 nm (Gu et al., 2020). The pseudo first order Hp-Hb/metHb binding rate constant was calculated by fitting the fluorescence intensity to a monoexponential equation. The pseudo first-order rate constant was then used to determine the bimolecular rate constant via linear regression with the metHb/Hb concentration as the dependent variable.

Haptoglobin-Binding via Size Exclusion HPLC (SEC-HPLC) Analysis. The molecular weight (MW) distribution of Hp-metHb mixtures was estimated via SEC-HPLC on an Acclaim SEC 1000 column (ThermoFisher Scientific, Waltham, MA). The binding capacity of haptoglobin (Hp) to Hb/metHb was analyzed by incubating a fixed concentration of Hp with Hb/metHb samples at different concentrations (1.25 μM, 2.5 μM, 5 μM, 10 μM, and 20 μM, Hb tetramer basis). All samples were analyzed via SEC-HPLC at a flow rate of 0.35 mL/min in the mobile phase (PB, 50 mM, pH 7.4). To determine the composition of Hp-metHb mixtures containing Hp-metHb complex, metHb tetramers (α₂β₂), dimers (αβ) and monomers (α/β), a deconvolution analysis was conducted based on the elution time at λ=413 nm of each pure species.

Methemoglobin (MetHb) Level. The metHb level of metbHb and methHb was measured using the cyanomethemoglobin assay as previously described in the literature (Drabkin, DL; Austin, 1935).

Storage Stability Analysis. The storage stability of bHb, hHb, metbHb and methHb was analyzed via analytical SEC-HPLC by incubating metHb samples at different concentrations (1.25 μM, 2.5 μM, 5 μM, 10 M, and 20 μM, heme basis) with 0.1 M PBS solution (pH 7.4) at 4° C. for one week. To determine the composition of denatured metHb mixtures containing metHb tetramers, dimers and monomers, a deconvolution analysis was performed based on the elution time of each pure species. To quantitively evaluate the dimerization equilibrium of Hb, the tetramer-dimer dissociation constant (K_{d}) was derived as follows:

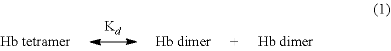

$${K}_{d} = \frac{{[ D ]}^{2}}{[ T ]} = \frac{{[ 2 ( [ H ] - [ T ] ) ]}^{2}}{[ T ]} = \frac{{4 [ H ] [ 1 - T \% ]}^{2}}{[ T ] / [ H ]} = \frac{{4 [ H ] [ 1 - T \% ]}^{2}}{T \%} \qquad ( 2 )$$

$${log}_{10} ( 1 / {K}_{d} ) = {log}_{10} ( \frac{T \%}{{4 [ 1 - T \% ]}^{2}} ) - {log}_{10} [ H ] \qquad ( 3 )$$

where [H] is the initial concentration of Hb/metHb tetramer, [T] is the concentration of Hb/metHb tetramers, [D] is the concentration of Hb/metHb αβdimers and T % is defined as the ratio [T]/[H]. Thus, a plot of

$${log}_{10} ( \frac{T \%}{{4 [ 1 - T \% ]}^{2}} )$$

with respect to log₁₀[H] yielded an intercept of log₁₀(1/K_{d}) as shown in Equation 3 (Manning et al., 2008; Sawicki & Gibson, 1981).

Statistical Analysis. A t-test was performed to study the variances among different groups of data, and a p-value (<0.05) demonstrates a significant difference.

### Results and Discussion

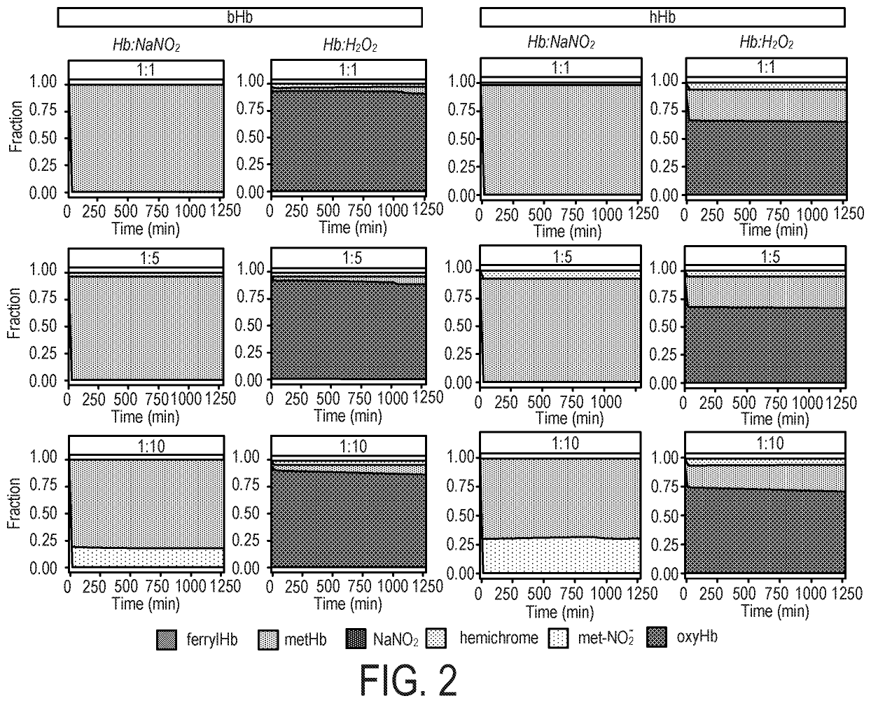

Molar Ratio of Hb (Heme Basis) to Oxidization Agent. In this initial study, two different oxidization agents (NaNO₂ and H₂O₂) were tested within a 21 hour window to determine the optimal molar ratio of Hb:oxidization agent, which led to the highest yield of metHb. The optimal oxidization agent and molar ratio of Hb:oxidization agent was then used for subsequent large-scale synthesis of metHb. FIG. 2 shows the composition of metHb mixtures prepared by reacting three different molar ratios of Hb:oxidization agent (1:1, 1:5, and 1:10). As shown in FIG. 2, the oxidation of both bHb/hHb at 1:1 and 1:5 molar ratios of Hb:NaNO₂ displayed rapid conversion of Hb to metHb, which is consistent with previously reported studies (Keszler, Piknova, Schechter, & Hogg, 2008). Approximately ˜95% of oxyhemoglobin (oxyHb) was converted to metHb within the first 30 mins of the reaction with negligible formation of hemichrome, suggesting high conversion to metHb with minimal side products.

For NaNO₂ oxidation of both bHb and hHb at a molar ratio of 1:5 in FIG. 2, a slightly higher fraction of hemichrome was produced in comparison to a molar ratio of 1:1, which can be attributed to the excess NaNO₂ in solution. NO₂⁻ most likely converts the high-spin metHb to low-spin hemichrome due to ligation of the distal histidine residue in the heme pocket (Schwab, Stamler, & Singel, 2010; Svistunenko et al., 2000; Welbourn, Wilson, Yusof, Metodiev, & Cooper, 2017). At a molar ratio of 1:10, it is interesting to note that adding more NaNO₂ did not lead to substantial formation of hemichrome, but instead led to the formation of a large fraction of metHb-NO₂. This can be explained by the dramatic pH increase in solution due to excess NaNO₂, which impeded hemichrome formation and facilitated NO₂⁻ binding to metHb (Sugawara et al., 2003). Oxidation at molar ratios of 1:1 and 1:5 yielded relatively high conversion of Hb to metHb (97.6% and 97.8% for bHb and hHb respectively) and negligible side products in comparison to that at a molar ratio of 1:10 (˜87.8% and 70.1% for bHb and hHb respectively). For Hb:H₂O₂ molar ratios of 1:1, 1:5, and 1:10, the final concentration of metHb was found to be 6.4%, 7.9% and 10.6% for bHb (FIG. 2), respectively. In FIG. 2, relatively higher conversions (29.1%, 28.7% and 23.7%) of hHb to methHb at various Hb:H₂O₂ molar ratios (1:1, 1:5, and 1:10) were observed due to the initially higher metHb level of hHb in comparison to bHb. Unfortunately, the final yield of metHb using H₂O₂ was still significantly lower than that using NaNO₂, indicating that H₂O₂ exhibited a weaker ability to convert Hb into metHb in comparison to NaNO₂. Additionally, H₂O₂ could induce chemical modification of the Hb protein structure, which might interfere with the binding between metHb and Hp (Vallelian et al., 2008). Taken together, H₂O₂ is not an ideal oxidization agent for the synthesis of metHb. Thus, in this study, NaNO₂ was used as the oxidization agent for scaleup synthesis of metHb. To further reduce the reaction time in the large scale metHb synthesis scheme, a Hb:NaNO₂ molar ratio of 1:5 was used for both metbHb and methHb synthesis.

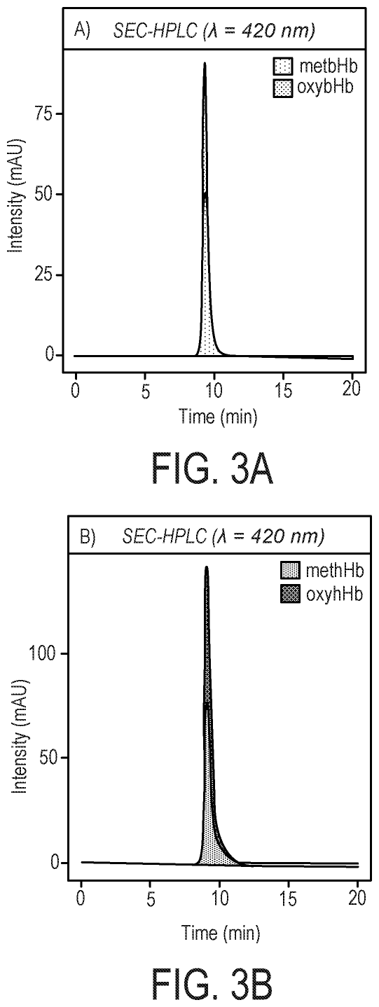

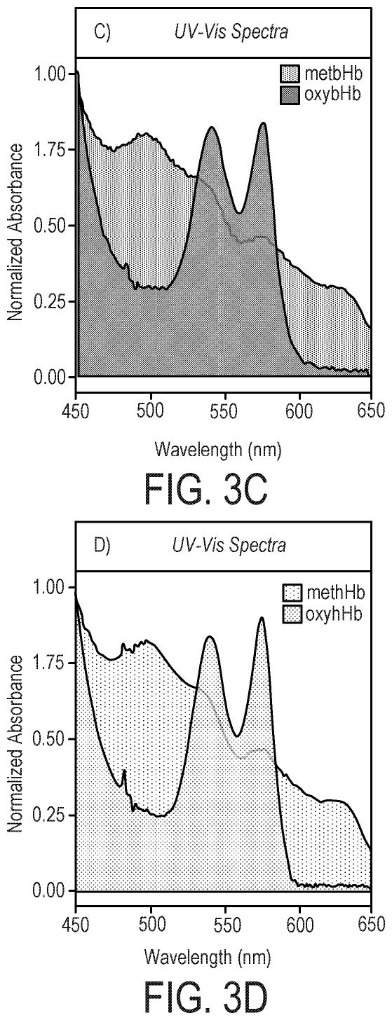

Molecular Weight. FIGS. 3A and 3B show the SEC-HPLC chromatogram of bovine/human oxyHb (oxybHb/oxyhHb), and freshly synthesized metHb (1 mg/mL, heme basis), which both eluted at the same time (9.29 mins). The molecular weight (MW) of oxybHb/oxyhHb and metbHb/methHb was estimated to be ˜64 kDa, which is consistent with the theoretical MW (Meng et al., 2018). Additionally, a single sharp peak was observed for both metbHb and methHb, indicating the high purity of the final product. The decreasing intensity of metHb compared to Hb at the same concentration was observed for both metbHb and methHb, which can be attributed to the left-shifted Soret peak of metbHb from 413 nm to 405 nm (Meng & Alayash, 2017). To further confirm the presence of metbHb/methHb, the full UV-visible spectra of metbHb and methHb were extracted from the SEC-HPLC chromatogram and displayed in FIGS. 3C and 3D. Four distinct peaks (505 nm, 540 nm, 575 nm and 629 nm) were observed in the Q-band region, which aligns well with the literature spectra of metHb (Cruz-Landeira, Bal, Quintela, & López-Rivadulla, 2002).

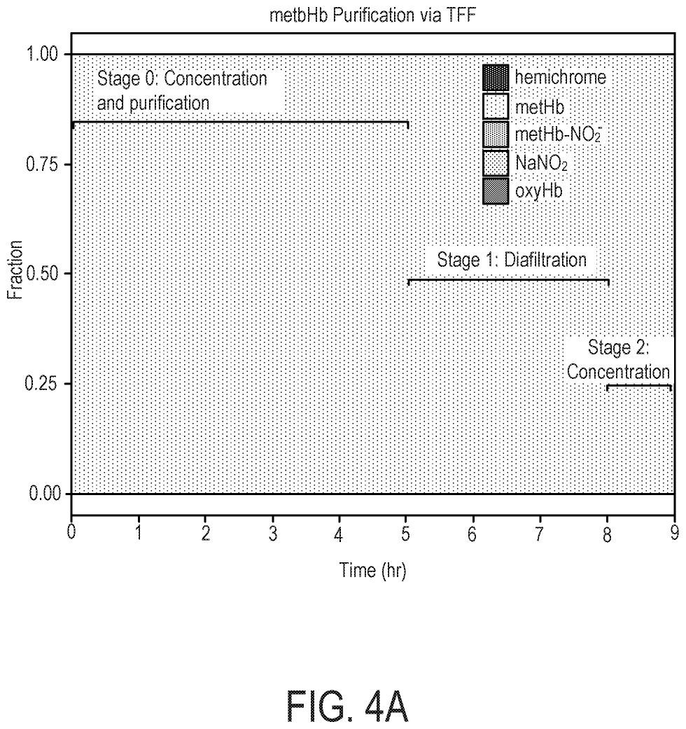

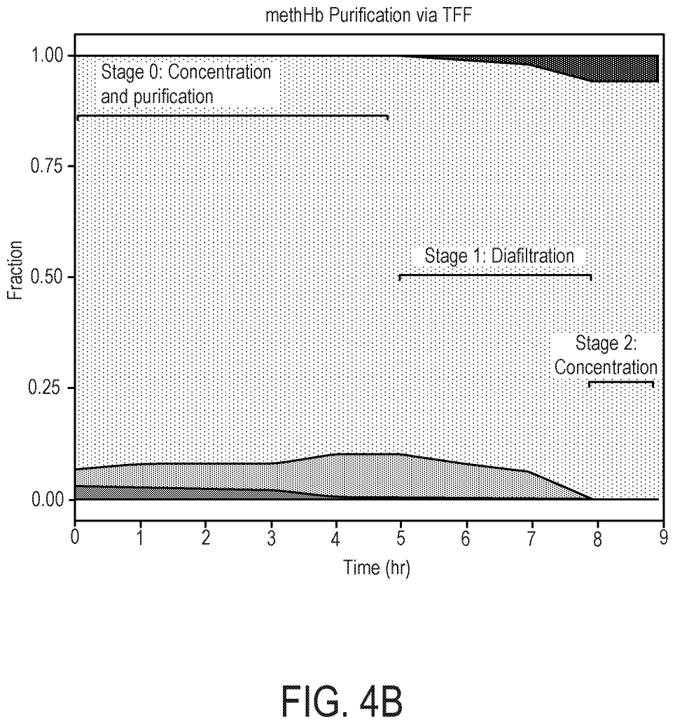

Composition of Hb Species During TFF. To evaluate the composition of metbHb/methHb mixtures as a function of TFF processing time after large scale synthesis, samples were collected every hour during the TFF process. The fraction of multiple Hb species including hemichrome, oxyHb, metHb, ferrylHb, metHb-NO₂⁻, and NaNO₂ was determined via spectra deconvolution using the Alchromy program. In FIG. 4A, a mixture consisting of almost pure metHb can be observed at all stages during the TFF metbHb purification process, which is consistent with results from the small-scale oxidization agent experiments. For TFF methHb purification in FIG. 4B, the composition changed at different stages during the purification process. At the beginning of stage 0, a mixture of 93.4% metHb, 2.8% oxyHb and 3.8% metHb-NO₂⁻ was observed which could be due to the initially higher metHb level of hHb (˜5%) in comparison to bHb (0.9%). The initially high metHb level of hHb is attributed to the fact that the hHb was purified from expired human RBCs, which possess a higher starting metHb level versus hHb derived from fresh human RBCs or RBCs used before the expiration date. At the end of stage 0, all oxyHb was converted to metHb-NO₂⁻ likely due to the presence of residual NaNO₂ in solution. After diafiltration was initiated (Stage 1), a dramatic decrease in the fraction of metHb-NO₂⁻ was observed at the end of the diafiltration process. This can be explained by the increased conversion of metHb-NO₂⁻ into metHb by removal of the residual NO₂⁻ via diafiltration on the 50 kDa HF module, demonstrating the importance of the diafiltration process using TFF in removal of excess reagents.

MetHb-Haptoglobin Binding Kinetics. The human haptoglobin (Hp) used in this study consisted of a mixture of phenotypes Hp2-1 and Hp2-2, which functions as a Hb scavenger to inhibit oxidation-mediated reactions elicited by the iron atom in Hb. Hp binds to Hb to form the Hp-Hb complex and removes it from the systemic circulation via CD163 receptor mediated endocytosis into macrophages and monocytes (Etzerodt & Moestrup, 2013). To determine the potential for metbHb to be cleared via CD163 mediated endocytosis, the ligand-binding kinetics of Hp with bHb/metbHb was monitored by rapidly mixing Hp with bHb/metbHb at various concentrations.

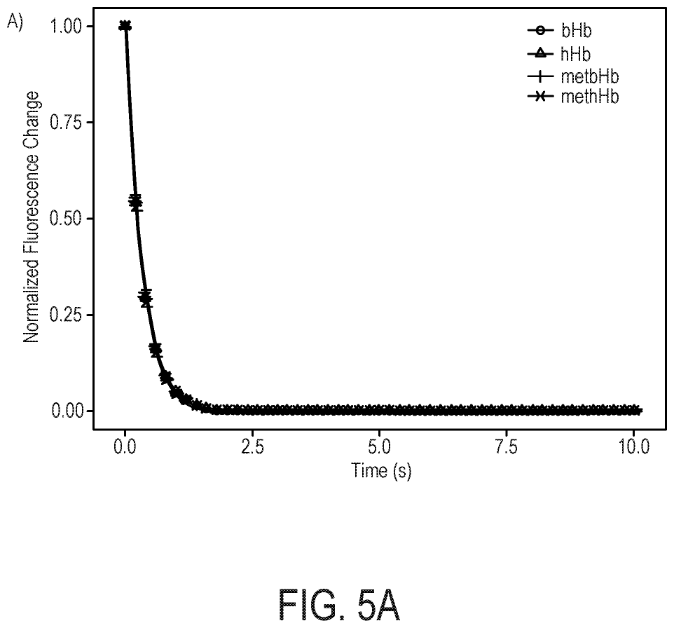

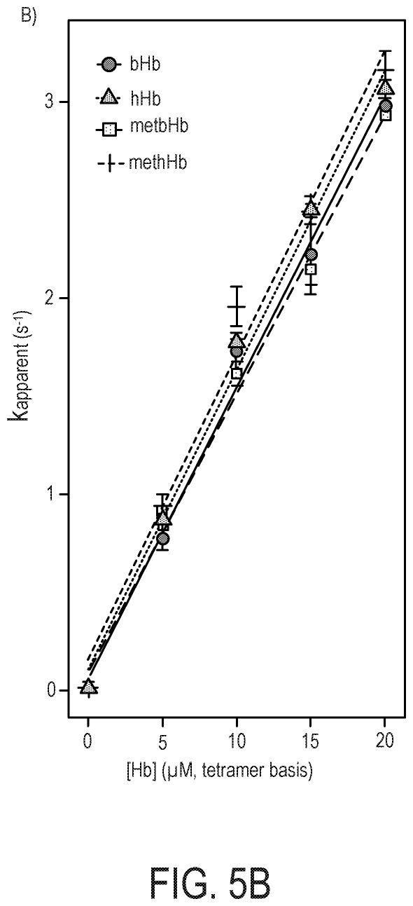

In FIG. 5A, the kinetics of Hp-bHb/metHb binding is shown, where metbHb was found to quench a similar number of Hb-binding sites in Hp in comparison to non-oxidized bHb. To determine the pseudo first order binding rate constants, kinetic traces were fit to a monoexponential equation (FIG. 5B). To calculate the 2^{nd} order (bHb/metbHb)-Hp binding rate constant (k_{Hp-Hb}), a linear fit to the data in FIG. 5B to determine the slope of the pseudo first order reaction rate constant as a function of metbHb/bHb concentration. Overall, k_{Hp-Hb} of metbHb (0.140±0.003 μM⁻¹ s⁻¹) exhibited no significant difference compared to unmodified bHb (0.147 ±0.006 μM⁻¹ s⁻¹), methHb (0.156 ±0.003 μM⁻¹ s⁻¹), and unmodified hHb (0.154±0.003 μM⁻¹ s⁻¹).

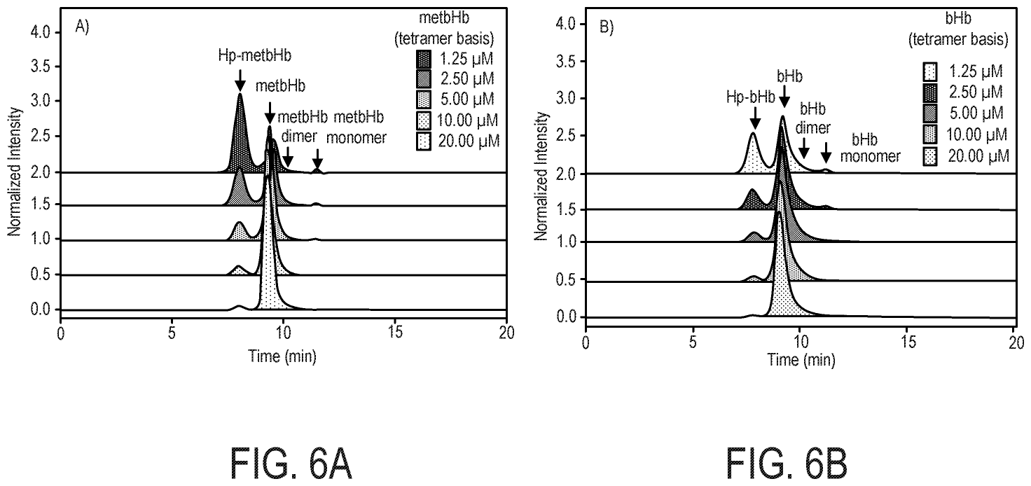

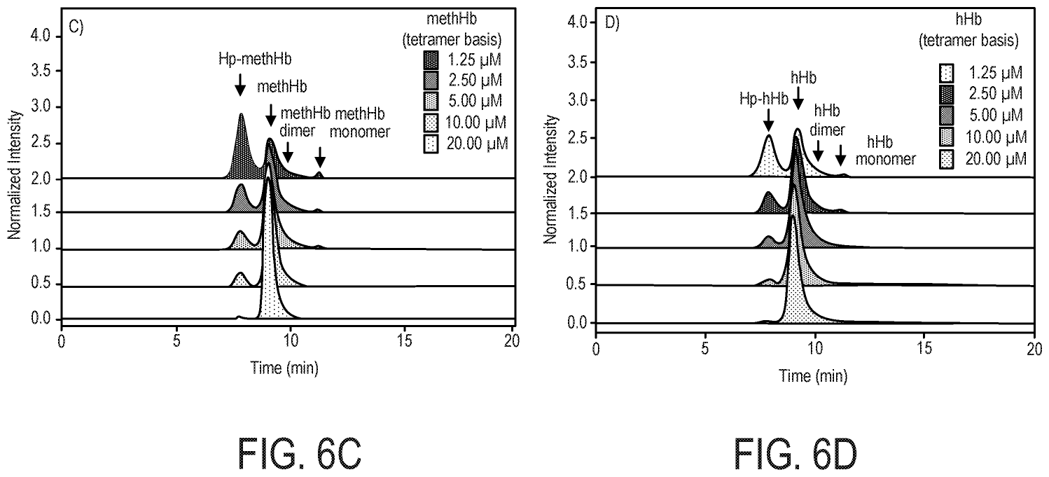

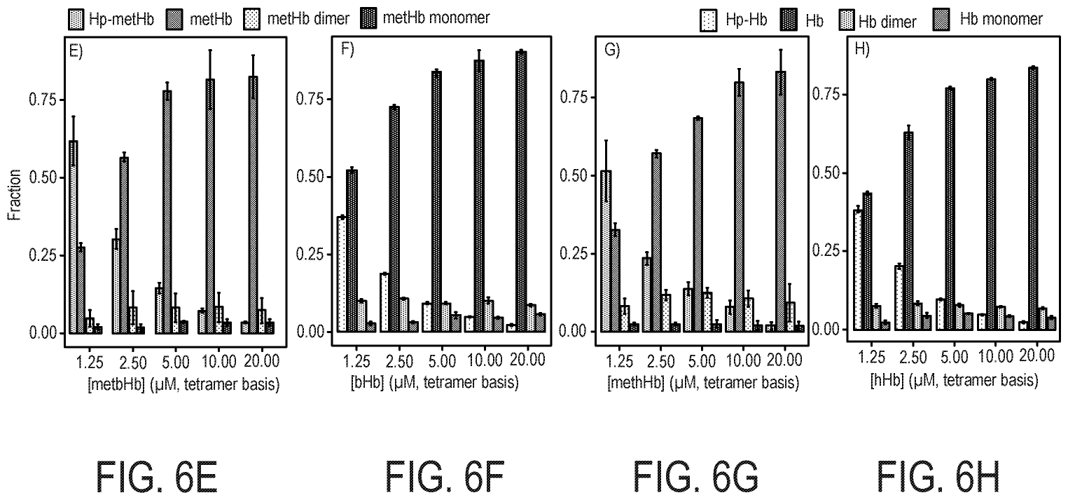

MetHb-Haptoglobin Binding via SEC-HPLC Analysis. As shown in FIGS. 6A and 6C, the molar fraction of Hp binding to metbHb/methHb was measured via analytical SEC-HPLC. The Hp mixture purified in our lab contained Hp 2-1 (˜200 kDa) and Hp 2-2 (˜400 kDa). To analyze the Hp-metHb complex, metbHb/methHb at different molar concentrations were initially mixed with excess Hp (2:1 Hp:metHb) as previously described in the literature (Belcher, Cuddington, Martindale, Pires, & Palmer, 2020). In comparison to Hp binding of native Hb, both bHb and hHb were prepared using the same protocol. A deconvolution program was then used to determine the molar fraction of each species including Hp-metHb complexes, metHb tetramers (α₂β₂), metHb dimers (αβ), and metHb monomers (α/β globin). The elution time of Hp-metHb was found to be ˜8.02 min, which corresponds to a MW of ˜400-500 kDa. It is evident that decreasing the molar concentration of metHb resulted in increased binding of Hp to metHb (from 0.78 μM to 0.87 μM), as anticipated. Hp binding facilitates dimerization of both metbHb and methHb, which led to a substantial reduction in the amount of tetrameric metbHb (from 82.4±6.9% to 27.6 ±1.3%) and methHb (from 83.2 ±7.3% to 32.6 ±2.0%) in solution (FIG. 6, panels A and C). Interestingly, both metbHb and methHb exhibited higher Hp binding capacity in comparison to bHb and hHb (FIG. 6, panels B and D) at relatively low concentrations (<5 μM, heme basis). This can be explained by the facilitated dimerization elicited by reducing the Hb concentration in solution.

In FIG. 6, panels E and G, no significant difference was observed between the metbHb/methHb fraction at concentrations of 5 μM and 10 μM, and 10 μM and 20 μM (Hb tetramer basis), indicating that the Hp binding equilibrium was not significantly affected by molar concentrations of Hb >5 μM. In the range 0-5 μM Hb, Hp-metHb binding was increased by lowering the metHb concentration, which was also observed for bHb/hHb in FIG. 6, panels F and H. Taken together, this indicates that the synthesized metHb can be potentially cleared from the systemic circulation via binding to Hp in a similar manner to Hb. The difference between the binding equilibrium of Hp-metHb and Hp-Hb can only be observed at metHb/Hb molar concentrations <5 μM.

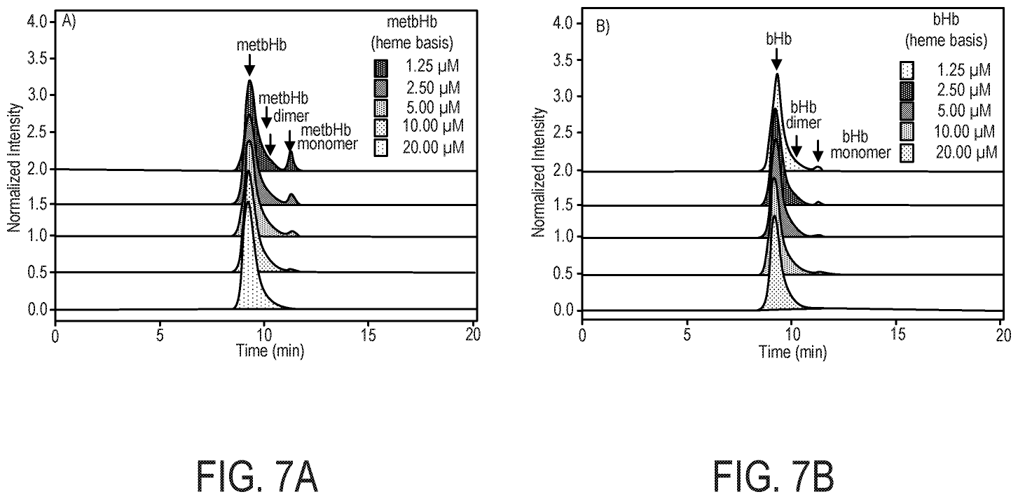

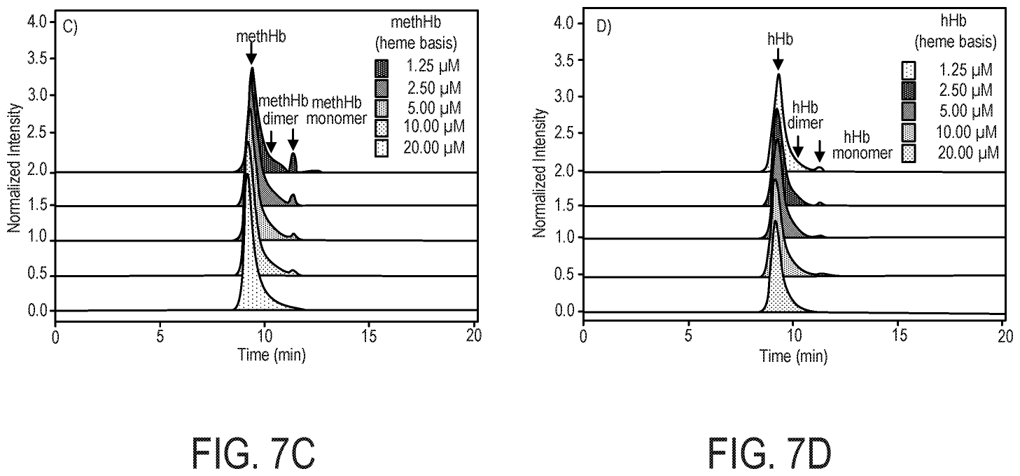

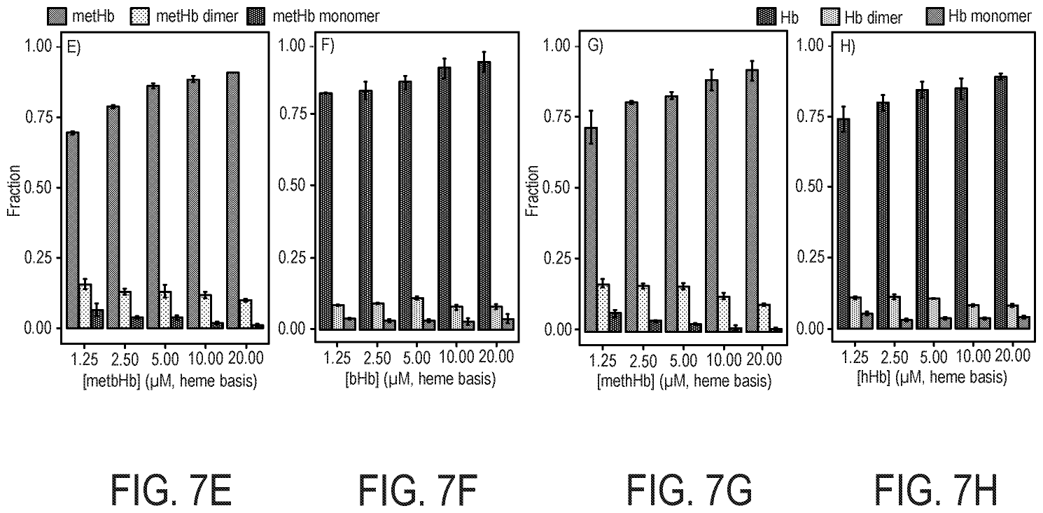

Storage Stability. In FIG. 7, panels A and C, the effect of the molar concentration of metbHb/methHb on its ability to dimerize into us dimers was examined by analytical SEC-HPLC. In this study, a serial dilution was performed to prepare metHb solutions at molar concentrations of 1.25 μM, 2.5 μM, 5 μM, 10 μM, and 20 μM (heme basis) after incubation in PBS (0.1 M, pH 7.4) at 4° C. for one week. In comparison to the stability of metbHb/methHb, native bHb/hHb was prepared following the same protocol (FIG. 7, panels B and D). To determine the molar fraction of each metHb species including metHb tetramers (α₂β₂), metHb dimers (αβ), and metHb monomers (α/β globin), a deconvolution analysis was conducted as previously described. An increasing molar fraction of metHb dimers (from 9.5 ±0.3% to 15.8 ±1.7%) and monomers (from 1.2 ±0.7% to 6.6±2.2%) was observed when lowering the molar concentration of metbHb from 20 μM to 1.25 μM (heme basis). Akin to metbHb, the fraction of methHb dimers increased from 9.4±0.6% to 13.7±1.3%, and from 1.1±0.8% to 6.7±0.9% for metHb monomers, upon dilution. In comparison to metbHb/methHb, slight Hb dissociation was observed for native bHb/hHb upon dilution (FIG. 7, panels B and D).

In FIG. 7, panels E and G, a negative correlation was observed between the fraction of dissociated metHb species (dimers and monomers) and the molar concentration of metbHb/methHb, indicating that the metbHb/methHb dissociation process is facilitated by reducing the molar concentration of metHb in solution. Additionally, both metbHb/methHb dimers and monomers were lower than 18% even at the extremely low concentration (1.25 μM, heme basis) after storage at 4° C. for one week, demonstrating the high stability of the synthesized metHb.

In FIG. 7, panels F and H, less variance was observed among all groups of the bHb/hHb tetramer at various concentrations in comparison to methHb/methHb, indicating that native Hb is more stable than metHb. The oxidation of heme from the ferrous state (Fe²⁺) to the ferric state (Fe³⁺) accelerates the conversion of tetrameric metHb into dimeric metHb. In general, both metbHb and methHb possessed greater than 87% of metHb tetramers at a concentration of 20 μM (heme basis). Thus, negligible tetramer dissociation can be anticipated when used in vivo at relatively high protein concentrations.

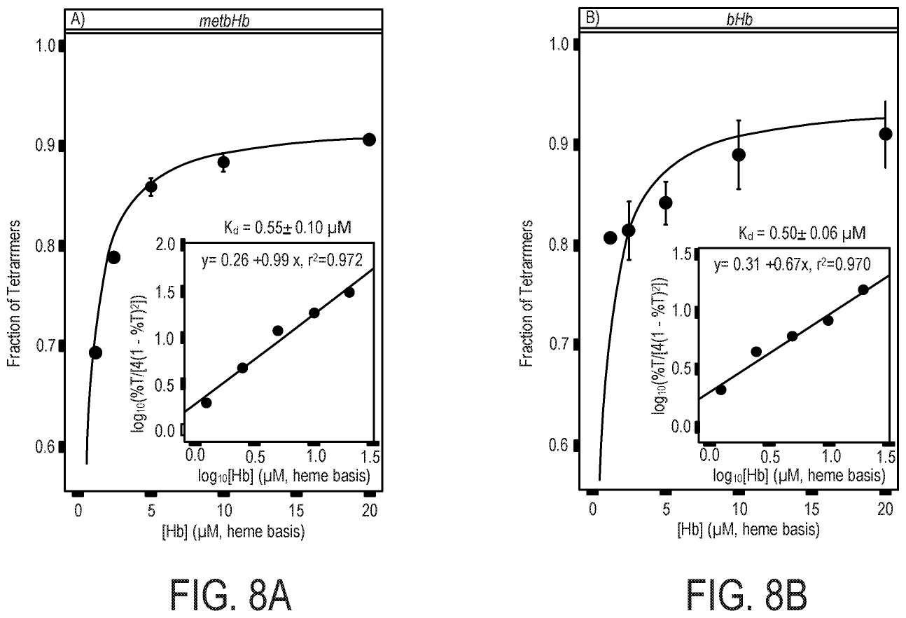

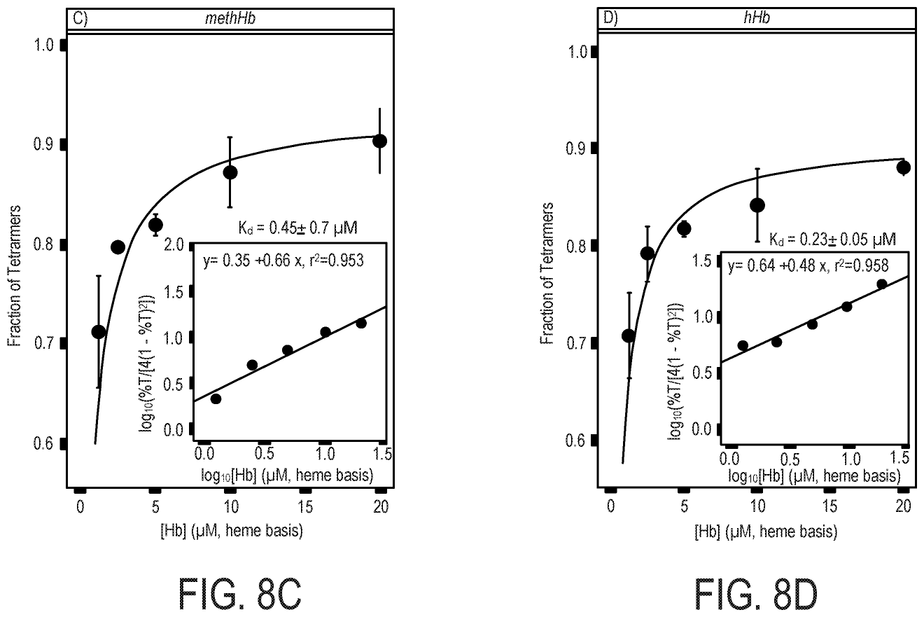

In FIGS. 8A and 8C, the fraction of metbHb/methHb tetramers was plotted as a function of metbHb/mehHb concentration, which yielded a hyperbolic profile. To determine the tetramer-dimer dissociation constant (K_{d}), a linear transformation was performed on the fraction of metbHb/methHb tetramers by using Equation 3, whose intercept yielded log(1/K_{d}). The K_{d} of metbHb (0.55 ±0.10 μM) and methHb (0.45 ±0.07 μM) can be further derived from the intercept value, which are comparable to previously reported values in the literature (Atha & Riggs, 1976; Griffon et al., 1998). In FIGS. 8B and 8D, it was found that native bHb and hHb possessed a K_{d} value of 0.50 ±0.06 μM and 0.23 ±0.05 μM, respectively, when incubated in PBS (0.1 M, pH 7.4) at 4° C. Although the K_{d} of metbHb/methHb seems to be slightly higher than native bHb/hHb, it is still on the same order of magnitude as the values for native Hb. In general, these results show that the conversion of heme from the ferrous state (Fe²⁺) to ferric state (Fe³⁺) has little effect on tetramer dissociation in the concentration range 1.25-20 μM, (heme basis) in PBS solution (pH 7.40).

### CONCLUSION

In this example, the optimal molar ratio of Hb:NaNO₂ was identified to synthesize metHb with high yield and purity using a batch reactor and TFF separation train. Additionally, no significant difference was found between the second order binding rate constant of Hp to metHb/Hb. Previous studies observed that elevated levels of nitrite in blood can oxidize the Hb inside RBCs, leading to methemoglobinemia. In this example, a negligible amount of nitrite was observed within the final metHb product, indicating the potential safety of using metHb as a suitable control material in small animal models. Future work will include evaluation of the safety and stability of this material in vivo. Taken together, both the metbHb and methHb synthesized in this example were stable at 4° C. in PBS (0.1 M, pH 7.4) for at least one week. The Hp-binding kinetics and equilibria of metbHb/methHb were comparable at metHb concentrations ranging from 5-20 μM (Hb tetramer basis). The difference between the Hp-binding equilibria of metHb and Hb can only be observed at extremely low concentrations (<5 μM, Hb tetramer basis).

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for preparing methemoglobin or polymerized methemoglobin, the method comprising:
(i) contacting a solution comprising ferrous hemoglobin or polymerized ferrous hemoglobin with an oxidizing agent under conditions effective to produce a solution comprising methemoglobin or polymerized methemoglobin; and
(ii) filtering the solution comprising methemoglobin or polymerized methemoglobin by ultrafiltration against a filtration membrane having a pore size that separates the protein from the oxidization agent and reaction byproducts, thereby forming a retentate fraction comprising the methemoglobin or polymerized methemoglobin and a permeate fraction comprising impurities.

2. The method of claim 1, where the ferrous hemoglobin or polymerized ferrous hemoglobin is from a mammalian, invertebrate, or recombinant source.

3. The method of claim 2, wherein the ferrous hemoglobin or polymerized ferrous hemoglobin is from a mammalian source.

4. The method of claim 3, wherein the ferrous hemoglobin or polymerized ferrous hemoglobin is from a human source.

5. The method of any of claims 1-4, wherein the polymerized ferrous hemoglobin is in the tense or relaxed quaternary state, or is in between these two quaternary states.

6. The method of any of claims 1-5, wherein step (i) comprises combining the ferrous hemoglobin or polymerized ferrous hemoglobin with the oxidizing agent in a reactor.

7. The method of any of claims 1-6, wherein the oxidizing agent comprises sodium nitrite, potassium nitrite, an inorganic peroxide, an organic peroxide, or any combination thereof.

8. The method of any of claims 1-7, wherein the filtration membrane is rated for removing solutes having a molecular weight less than the molecular weight of the methemoglobin or polymerized methemoglobin.

9. The method of any of claims 1-8, wherein the filtration membrane is rated for removing solutes having a molecular weight of from 1 to 750 kDa, such as from 1 to 500 kDa, from 1 to 250 kDa, from 1 to 100 kDa, from 1 to 50 kDa, or from 1 to 10 kDa.

10. The method of any of claims 1-9, wherein the ultrafiltration process comprises tangential flow filtration.

11. The method of any of claims 1-9, wherein the ultrafiltration process comprises cross-flow filtration.

12. The method of any of claims 1-11, wherein filtering step (ii) comprises buffer exchange.

13. The method of any of claims 1-12, wherein filtering step (ii) comprises continuous diafiltration or dialysis.

14. The method of claim 13, wherein the retentate fraction is spectroscopically monitored during the continuous diafiltration process to monitor the formation of methemoglobin or polymerized methemoglobin, the concentration of methemoglobin or polymerized methemoglobin, the concentration of an impurity, or any combination thereof.

15. The method of any of claims 1-14, wherein the oxidizing agent comprises sodium nitrite, potassium nitrite, or any combination thereof.

16. The method of claim 15, wherein step (i) comprises combining the ferrous hemoglobin or polymerized ferrous hemoglobin with the oxidizing agent at a molar ratio (heme basis) of from 2:1 to 1:10, such as from 1:1 to 1:5.

17. The method of any of claims 1-16, wherein step (i) comprises reacting the ferrous hemoglobin or polymerized ferrous hemoglobin with the oxidizing agent for from 1 minute to 24 hours, such as from 5 minutes to 8 hours or from 5 minutes to 4 hours.

18. The method of any of claims 1-17, wherein step (i) comprises reacting the ferrous hemoglobin or polymerized ferrous hemoglobin with the oxidizing agent at a temperature of from 1° C. to 40° C.

19. The method of any of claims 1-18, wherein the solution comprising ferrous hemoglobin or polymerized ferrous hemoglobin comprises an aqueous solution buffered at a pH of from 6 to 8.

20. The method of any of claims 1-19, wherein the methemoglobin or polymerized methemoglobin in the retentate fraction is stable at 4° C. in PBS (0.1 M, pH 7.4) for a period of at least 7 days.

21. A pharmaceutical composition comprising methemoglobin or polymerized methemoglobin prepared using the method defined by any of claims 1-20.

22. The composition of claim 21, wherein the methemoglobin is complexed with haptoglobin.

23. The composition of claim 22, wherein the methemoglobin is complexed with haptoglobin at a weight ratio of from 1:1 to 1:3 (methemoglobin:haptoglobin).

24. The composition of any of claims 22-23, wherein the haptoglobin has an average molecular weight of from 80 kDa to 1,000 kDa, such as from 100 kDa to 1,000 kDa.

25. The composition of any of claims 21-24, wherein the methemoglobin, the polymerized methemoglobin, or the methemoglobin complexed with haptoglobin is conjugated to a therapeutic or diagnostic agent.

26. The composition of claim 25, wherein the therapeutic or diagnostic agent comprises an imaging agent, such as an MRI contrast agent, a porphyrin-based imaging agent, and/or a phthalocyanine-based imaging agent, a porphyrin-based photodynamic therapy agent, a phthalocyanine-based photodynamic therapy agent, an agent to treat or prevent a disease or disorder associated with the overexpression of CD163, an agent to treat or prevent a disease which involves macrophages or monocytes, an anti-cancer agent, an anti-inflammatory agent, an agent that treats or prevents an infection, hydrogen sulfide, or a combination thereof.

27. The composition of any of claims 21-26, wherein the composition is administered as an immunotherapy targeting CD163+ macrophages and monocytes.

28. A population of particles comprising hemoglobin or polymerized methemoglobin prepared using the method defined by any of claims 1-20.

29. The population of particles of claim 28, wherein the methemoglobin is complexed with haptoglobin.

30. The population of particles of any of claims 28-29, wherein the population of particles has an average particle size of from 10 nm to 1 cm.

31. A method of treating cyanide or hydrogen sulfide poisoning in a subject in need thereof comprising administering the subject a therapeutically effective amount of the composition defined by any of claims 21-27.

32. A method for lowering the metabolism of a cell, tissue, or organ, the method comprising contacting the cell, tissue, or organ with a therapeutically effective amount of the composition defined by any of claims 21-27, wherein the composition comprises hydrogen sulfide bound to the methemoglobin, the polymerized methemoglobin, or the methemoglobin complexed with haptoglobin.

33. A method for lowering the metabolism of a subject, the method comprising administering to the subject a therapeutically effective amount of the composition defined by any of claims 21-27, wherein the composition comprises hydrogen sulfide bound to the methemoglobin, the polymerized methemoglobin, or the methemoglobin complexed with haptoglobin.
